(19) United States
(12) Patent Application Publication
(10) Pub. No.: US 2024/0099630 A1
(43) Pub. Date: Mar. 28, 2024

(54) Electrocardiogram Systems and Devices and Related Methods

(71) Applicant: Biotricity Inc., Redwood City, CA (US)

(72) Inventor: Waqaas Al-Siddiq, Redwood City, CA (US)

(73) Assignee: Biotricity Inc., Redwood City, CA (US)

(21) Appl. No.: 18/476,046

(22) Filed: Sep. 27, 2023

Related U.S. Application Data
(60) Provisional application No. 63/377,336, filed on Sep. 27, 2022.

(51) Int. Cl. A61B 5/282; A61B 5/257; A61B 5/304; A61B 5/339

(52) U.S. Cl. CPC A61B 5/282; A61B 5/257; A61B 5/304; A61B 5/339

(57) ABSTRACT

An ECG device may include a housing, a plurality of leads coupled with the housing, and couplers configured to attach the leads to electrodes. A marking switch may be coupled with the housing. The ECG device may be configured to monitor cardiac-related activity and record related data. The marking switch may be configured to record a heart-related event when engaged by a user. User interfaces on one or more computing devices may allow a clinician and/or a patient to view information related to the ECG device and its recorded data, as well as to facilitate communication between the ECG device and one or more servers and to associate symptoms with heart-related events. ECG systems include the ECG device and the one or more servers. ECG methods involve using the ECG device and ECG system in recording and storing heart-related data and allowing clinicians access to retrieve it for analysis.

### CROSS REFERENCE TO RELATED APPLICATIONS

This document claims the benefit of the filing date of U.S. Provisional Patent Application No. 63/377,336, entitled “Electrocardiogram Systems and Devices and Related Methods,” naming as first inventor Waqaas Al-Siddiq, which was filed on Sep. 27, 2022, the disclosure of which is hereby incorporated entirely herein by reference.

### BACKGROUND

### 1. Technical Field

Aspects of this document relate generally to electrocardiogram (ECG or EKG) systems and devices and related methods.

### 2. Background Art

Electrocardiogram devices exist in the art and are used to produce an electrocardiogram (ECG or EKG), which is a recording of the heart's electrical activity through repeated cardiac cycles. In some cases the ECG/EKG is an electrogram graphing electric activity of the heart by plotting voltage versus time, as measured using electrodes placed on the skin. In implementations these electrodes detect the small electrical changes that are a consequence of cardiac muscle depolarization followed by repolarization during each cardiac cycle (heartbeat). Changes in the normal ECG pattern occur in numerous cardiac abnormalities, and accordingly ECGs may assist and/or facilitate heart health monitoring and/or the diagnosis of various heart-related conditions.

### SUMMARY

In implementations, the techniques described herein relate to an electrocardiogram (ECG) device, including: a housing; a plurality of leads coupled with the housing and including couplers configured to attach to electrodes; and a marking switch coupled with the housing; wherein the ECG device is configured to monitor cardiac-related activity and record related data; and wherein the marking switch is configured to, when engaged by a user, record a heart-related event.

In implementations, the techniques described herein relate to an ECG device, wherein the ECG device includes no more than three leads.

In implementations, the techniques described herein relate to an ECG device, wherein the ECG device monitors the cardiac-related activity via no fewer than three channels.

In implementations, the techniques described herein relate to an ECG device, wherein the couplers include snap connectors.

In implementations, the techniques described herein relate to an ECG device, wherein the leads are rigid and extend away from the housing.

In implementations, the techniques described herein relate to an ECG device, further including a status indicator light configured to visually indicate a plurality of statuses of the ECG device.

In implementations, the techniques described herein relate to an ECG device, wherein the status indicator light is housed at least partially within the housing, wherein the marking switch is at least partially translucent, and wherein the status indicator light visually indicates the plurality of statuses by shining through the marking switch.

In implementations, the techniques described herein relate to an ECG device, wherein the electrodes include one of: rectangular electrodes having largest dimensions of no more than four inches by four inches; and circular electrodes having diameters of no more than four inches.

In implementations, the techniques described herein relate to an ECG device, wherein the ECG device is configured to be secured to the user using only adhesion of the electrodes to skin of the user.

In implementations, the techniques described herein relate to an electrocardiogram (ECG) system, including: an ECG device configured to monitor cardiac-related activity and record related data, the ECG device at least intermittently communicatively coupled with one or more servers through one or more computing devices, the ECG device including: a housing; and a plurality of leads extending from the housing and each including a coupler configured to releasably attach to an electrode; and one or more user interfaces accessible through one of a browser and a software application installed on the one or more computing devices, the one or more computing devices at least intermittently communicatively coupled with the one or more servers and with the ECG device, the one or more user interfaces including at least one of: an indicator configured to indicate a status of a communicative connection between the ECG device and the one or more servers; and an indicator configured to indicate a status of a communicative connection between the ECG device and the one or more computing devices.

In implementations, the techniques described herein relate to a system, wherein the one or more user interfaces further include an indicator configured to indicate a status of a heart-related study of a patient.

In implementations, the techniques described herein relate to a system, wherein the one or more user interfaces further include an indicator configured to indicate a connection status of an electrode.

In implementations, the techniques described herein relate to a system, wherein the ECG device further includes a marking switch coupled with the housing.

In implementations, the techniques described herein relate to a system, wherein the one or more user interfaces further include a symptoms user interface (e.g., user interface 154) displayed in response to user engagement of the marking switch.

In implementations, the techniques described herein relate to a system, wherein the symptoms user interface displays a list of symptoms and is configured to receive a selection of one or more of the listed symptoms.

In implementations, the techniques described herein relate to a system, wherein the ECG device is configured to upload recorded data, related to the cardiac-related activity, to the one or more servers through the communicative connection between the ECG device and the one or more computing devices.

In implementations, the techniques described herein relate to a system, wherein the ECG device includes no more than three leads and is configured to monitors the cardiac-related activity via no fewer than three channels.

In implementations, the techniques described herein relate to a method of use for an electrocardiogram (ECG) device, including: providing an ECG device configured to monitor cardiac-related activity and to record cardiac-related data, the ECG device including: a housing; a plurality of leads coupled with the housing and including snap couplers configured to attach to electrodes; and a marking switch coupled with the housing and configured to initiate recording of a heart-related event upon being engaged by a user; providing one or more user interfaces through one of a browser and a software application installed on one or more computing devices, the one or more user interfaces including one or more selectors configured to allow a clinician to: communicatively couple the one or more computing devices with one or more servers and with the ECG device; and initiate recording of the cardiac-related data by the ECG device.

In implementations, the techniques described herein relate to a method, further including, in response to a predetermined time period elapsing after initiating recording of the cardiac-related data, uploading the recorded cardiac-related data to the one or more servers through the one or more computing devices.

In implementations, the techniques described herein relate to a method further including using an ECG classifier, at least partially comprised in one of the one or more servers and the ECG device, to analyze the cardiac-related data and to determine and indicate one of the following: placement in time of an R peak of a heartbeat; a type for the heartbeat; existence of an arrythmia; existence of a heart block; a diagnosis; heart rate variability (HRV) statistics; ST segment statistics; and QTC segment statistics.

General details of the above-described implementations, and other implementations, are given below in the DESCRIPTION, the DRAWINGS, the CLAIMS and the ABSTRACT.

### BRIEF DESCRIPTION OF THE DRAWINGS

Implementations will be discussed hereafter using reference to the included drawings, briefly described below, wherein like designations refer to like elements. The drawings are not necessarily drawn to scale.

FIG. 1 is a top perspective view of an implementation of an electrocardiogram (ECG) device;

FIG. 2 is a bottom perspective view of the ECG device of FIG. 1;

FIG. 3 is a close-up top perspective view showing a lead of the ECG device of FIG. 1 being coupled to (or decoupled from) an electrode;

FIG. 4 is a top, side perspective view of the ECG device of FIG. 3 showing the lead coupled to the electrode;

FIG. 5 representatively illustrates example placement positions for the ECG device of FIG. 1 on male and female patients;

FIG. 6 shows an alternative placement position for the ECG device of FIG. 1 on a male with an incision site;

FIG. 7 is an example user interface implemented using the system of FIG. 12;

FIG. 8 is an example user interface implemented using the system of FIG. 12;

FIG. 9 is an example user interface implemented using the system of FIG. 12;

FIG. 10 is an example user interface implemented using the system of FIG. 12;

FIG. 11 is an example user interface implemented using the system of FIG. 12;

FIG. 12 is an example of an ECG system including the ECG device of FIG. 1;

FIG. 13 is a diagram representatively illustrating example methods of use of the ECG system of FIG. 12;

FIG. 14 is a diagram representatively illustrating example methods of use of the ECG system of FIG. 12;

FIG. 15 is a diagram representatively illustrating example methods of use of the ECG system of FIG. 12;

FIG. 16 is a diagram representatively illustrating example methods of use of the ECG system of FIG. 12;

FIG. 17 is a diagram representatively illustrating an example software design pattern for software of the ECG system of FIG. 12;

FIG. 18 is a diagram representatively illustrating an example software design pattern for software of the ECG system of FIG. 12;

FIG. 19 is a diagram representatively illustrating an example software design pattern for software of the ECG system of FIG. 12;

FIG. 20 is a diagram representatively illustrating example methods of use of the ECG system of FIG. 12;

FIG. 21A is a diagram representatively illustrating a first portion of an example architecture for software of the ECG system of FIG. 12;

FIG. 21B is an extension of the diagram of FIG. 21A and representatively illustrates a second portion of the example architecture for software of the ECG system of FIG. 12;

FIG. 21C is an extension of the diagram of FIG. 21B and representatively illustrates a third portion of the example architecture for software of the ECG system of FIG. 12;

FIG. 22 is a diagram representatively illustrating example methods of use of the ECG system of FIG. 12;

FIG. 23 is a diagram representatively illustrating example methods of use of the ECG system of FIG. 12;

FIG. 24 is a diagram representatively illustrating example methods of use of the ECG system of FIG. 12;

FIG. 25 is a diagram representatively illustrating example methods of use of the ECG system of FIG. 12;

FIG. 26 is a diagram representatively illustrating example methods of use of the ECG system of FIG. 12;

FIG. 27 is a diagram representatively illustrating example methods of use of the ECG system of FIG. 12;

FIG. 28 is a diagram representatively illustrating example methods of use of the ECG system of FIG. 12;

FIG. 29 is a diagram representatively illustrating example methods of use of the ECG system of FIG. 12;

FIG. 30 is a diagram representatively illustrating example methods of use of the ECG system of FIG. 12;

FIG. 31 is a diagram representatively illustrating example methods of use of the ECG system of FIG. 12;

FIG. 32 is an exploded top perspective view showing example elements of the ECG device of FIG. 1; and

FIG. 33A is a diagram representatively illustrating a first portion of example hardware elements, signals, and power lines of the ECG device of FIG. 1; and

FIG. 33B is an extension of the diagram of FIG. 33A and representatively illustrates a second portion of example hardware elements, signals, and power lines of the ECG device of FIG. 1.

### DESCRIPTION

Implementations/embodiments disclosed herein (including those not expressly discussed in detail) are not limited to the particular components or procedures described herein. Additional or alternative components, assembly procedures, and/or methods of use consistent with the intended electrocardiogram systems and devices and related methods may be utilized in any implementation. This may include any materials, components, sub-components, methods, sub-methods, steps, and so forth.

Implementations of electrocardiogram (ECG) systems and devices and related methods relate to systems, devices and methods for recording a heart's electrical activity.

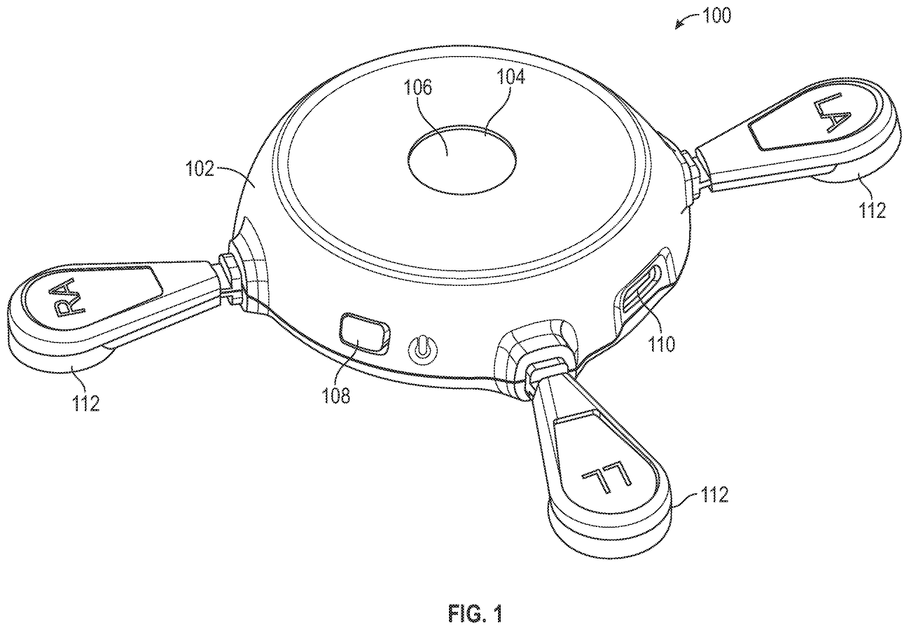

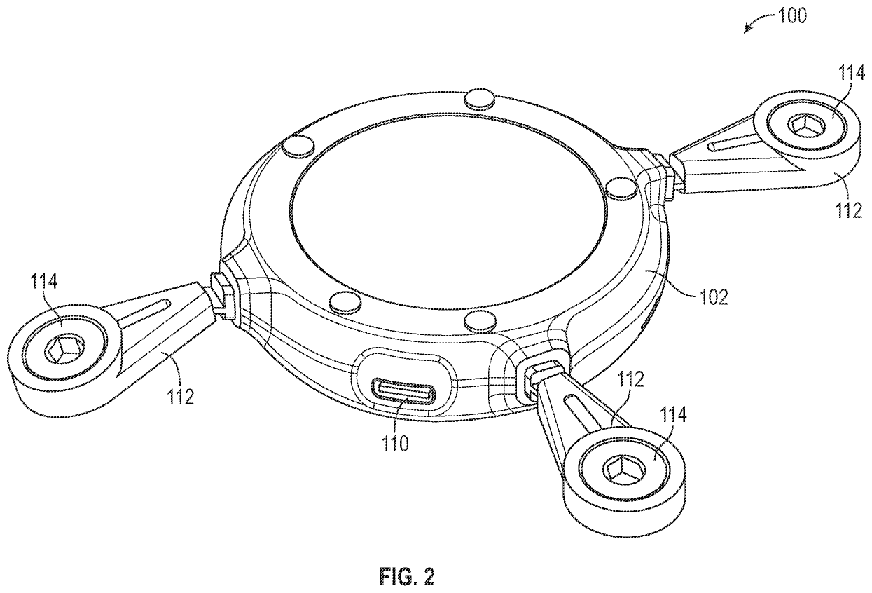

Referring now to FIGS. 1-2, an implementation of an ECG device 100 is shown. The ECG device includes a housing 102, a marking switch 104 with a status indicator light 106, a power switch 108, a charging port 110, and a plurality of leads 112 each having a snap connector 114. In the drawings the marking switch and the power switch are buttons, but in other implementations they could be any other type of electrical toggles/switches. In implementations the housing is formed of one or more polymers. The leads include LA, LL, and RA markings and the snap connectors 114 are seen to be female connectors, though they could be male connectors in other implementations. The leads electrically couple one or more electrodes with components internal to, or otherwise electrically coupled with, the ECG device.

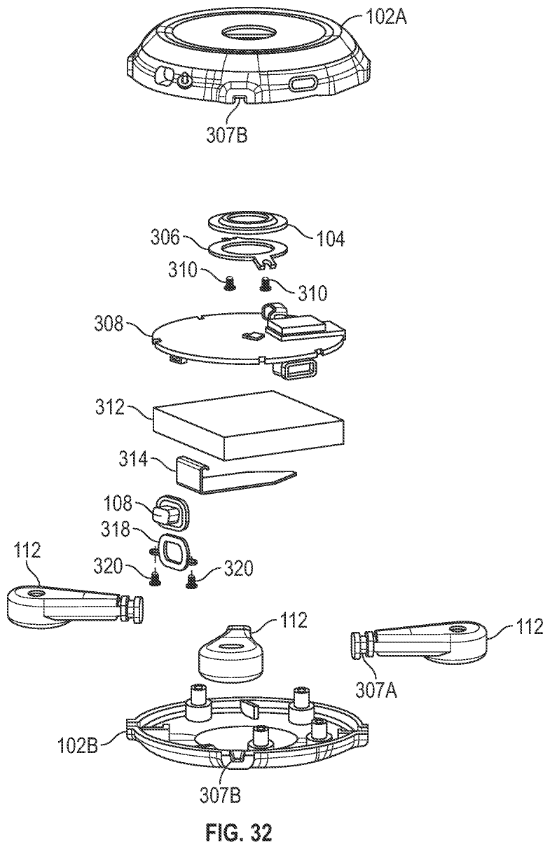

For example, referring to FIG. 32, the housing may include a first housing portion 102A and a second housing portion 102B which may be secured to one another using adhesive(s), threaded couplers such as screws, clamp elements, or any other securing mechanism. The marking switch, power switch, and leads are visible. Each lead is seen to have a recess 307A which encircle its narrow end to form a slot and which allows the lead to be secured within a corresponding projection 307B jointly formed by the housing portions which sits within the encircling slot, albeit somewhat loosely by the projections being sized somewhat smaller than the recesses to allow some give. This allows the leads to be secured to the housing but, nevertheless, have some allowable motion relative thereto. This can help the leads to be more comfortably and/or reliably positioned on a patient regardless of differences in chest or body shapes/sizes etc. In other cases the recess and projection are configured for a tight fit so that the leads to not have allowable motion relative to the housing.

A coupler 306 couples with the marking switch 104 and in some cases secures the marking switch 104 to a printed circuit board or PCB 308 using couplers 310 which pass through receivers/slots or through-holes (which may be threaded) of coupler 306. Couplers 310 may be threaded couplers such as screws or bolts. In some cases coupler 306 is adhesively secured to marking switch 104 and/or is otherwise secured thereto and holds the marking switch 104 in a position in which it is electrically coupled with the PCB 308. In some cases the coupler 306 and/or couplers 310 electrically couple the marking switch 104 with the PCB 308. The PCB 308 includes the charging port 110 and may include other elements such as hardware, one or more processors, memory for implementing software elements, and so forth to implement the functions described herein. A status indicator light of a different variety than in FIG. 1 is seen projecting atop the marking switch. In implementations the opening in the first housing portion 102A through which the marking switch 104 is exposed has a diameter smaller than a largest diameter of marking switch 104, thus allowing marking switch 104 to be activated/pushed by the user to toggle it but also allowing it to be retained within housing 102. One or more biased members, such as a leaf or coil spring, could be used to bias the marking switch 104 to an outward position. Depressing the marking switch 104 may complete an electrical circuit (such as by one or more electrical contacts on an underside of marking switch 104) which may function in conjunction with electrical elements of PCB 308 and/or software/hardware of ECG device 100 to allow electrical toggling on/off and/or between different settings when marking switch 104 is depressed once or multiple times in sequence or multiple times within a predetermined time frame, or using a long press, etc.

A rechargeable battery 312 is included and a conductive tape member 314 electrically couples the battery with the power switch 108 to allow power from the battery to be toggled on and off. The battery is electrically coupled with the charging port to allow its charging. The conductive tape member 314 could be replaced with wiring or some other element to electrically couple the power switch 108 with the battery.

A coupler 318 couples with the power switch 108 and in some cases secures the power switch 108 to the housing 102 using couplers 320 which pass through receivers/slots or through-holes (which may be threaded) of coupler 318. Couplers 320 may be threaded couplers such as screws or bolts. In some cases coupler 318 is adhesively secured to power switch 108 and/or is otherwise secured thereto and holds the power switch 108 in a position in which it is electrically coupled with the battery (through the conductive tape member 314 or by some other intermediary) to allow toggling of the power to the PCB 308 and/or other elements of the ECG device 100. In some cases the coupler 318 and/or couplers 320 electrically couple the power switch 108 with the battery. In implementations the opening in the first housing portion 102A through which the power switch 108 is exposed has a diameter or size smaller than a largest diameter of power switch 108, thus allowing power switch 108 to be activated/pushed by the user to toggle it but also allowing it to be retained within housing 102. One or more biased members, such as a leaf or coil spring, could be used to bias the power switch 108 to an outward position. Depressing the power switch 108 may complete an electrical circuit (such as by one or more electrical contacts on an underside or backside of power switch 108) which may function in conjunction with electrical elements of PCB 308 and/or software/hardware of ECG device 100 to allow electrical toggling on/off and/or between different settings when marking switch 104 is depressed once or multiple times in sequence or multiple times within a predetermined time frame, or using a long press, etc.

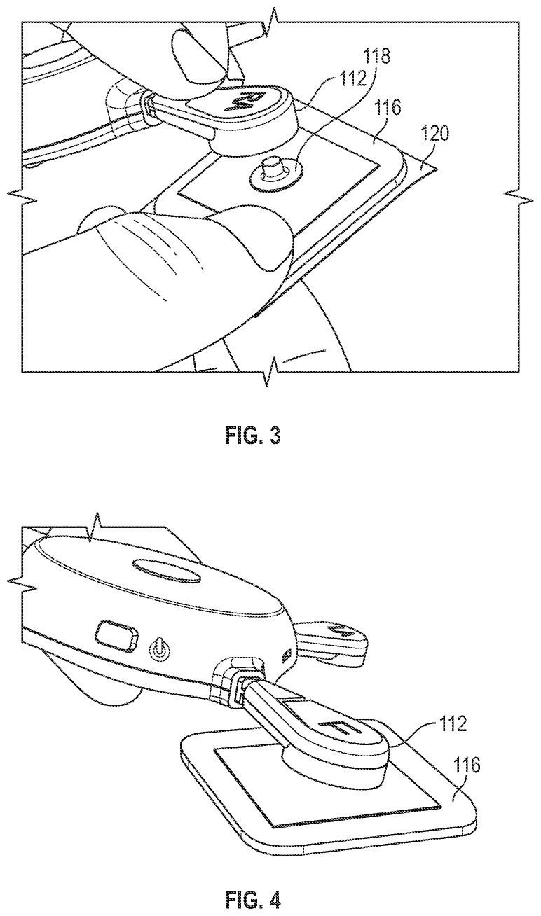

Referring now to FIG. 3, the RA lead is shown being attached to an electrode 116. The electrode includes a snap connector 118, which in the drawing is a male connector configured to selectively/releasably couple with the lead's female connector, though in implementations this configuration could be reversed. A backing 120 is adhesively coupled with the electrode to preserve adhesive on the back of the electrode until the electrode is placed onto a user's skin. The adhesive on the back of the electrode helps the electrode to stay in a secure position on the user's skin. FIG. 4 shows the LL electrode attached to an electrode with the backing 120 removed and ready to be adhered to a user's skin.

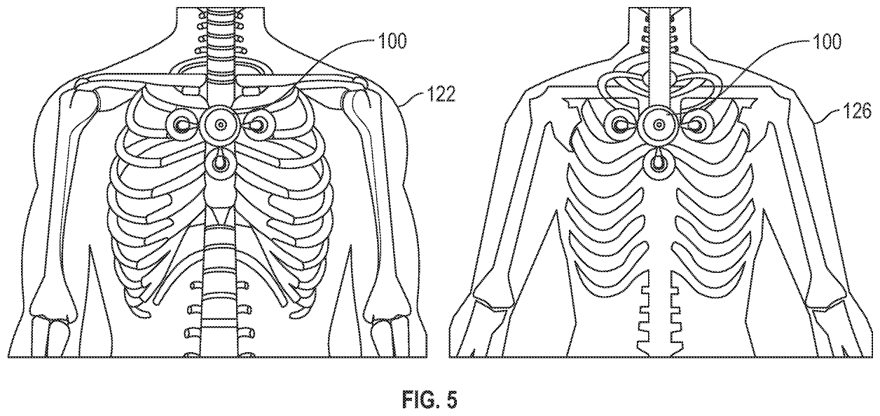

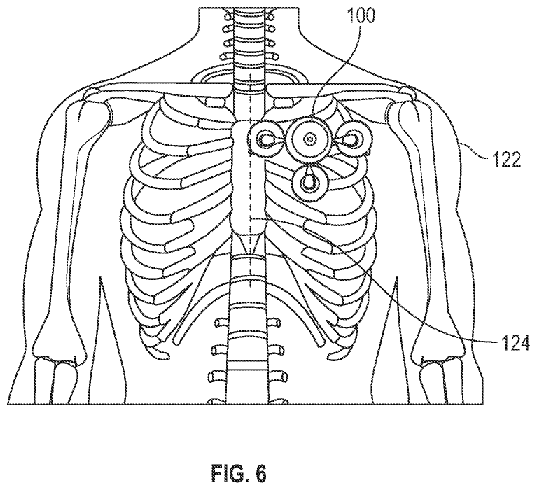

FIG. 5 shows example placement positions for the electrodes and for ECG device 100 on a male 122 and a female 126. The placement position shown for the female 126 can alternatively have a position that is slightly lower and slightly to the right (relative to the page) than what is shown in FIG. 5. Other positions can also be supported for both male and female placement. The electrodes in FIG. 5 have a circular configuration, as opposed to the rectangular configuration of the electrodes of FIGS. 3-4. Any size/shape of electrodes may be used with the ECG device. FIG. 6 shows alternative placement positions for the electrodes and for the ECG device 100 on a male 122 having an incision site 124.

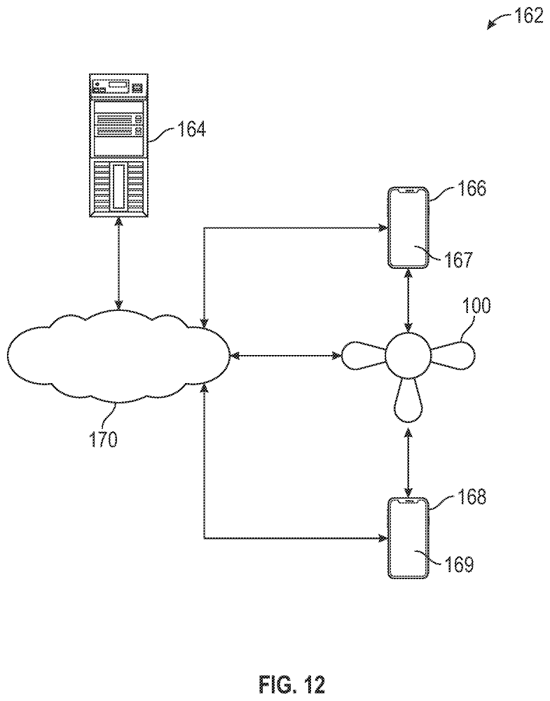

Referring now to FIG. 12, a representative example of an ECG system 162 is shown. The ECG system includes one or more servers 164, the ECG device 100, and two computing devices 166 and 168. Computing device 166 is a clinician computing device and computing device 168 is a patient computing device. Device 166 includes a display 167 and device 168 includes a display 169. The computing devices are communicatively coupled with the ECG device, such as through a BLUETOOTH or other wireless communicative coupling (though in implementations they could be communicatively coupled with a wired coupling). The computing devices may also be communicatively coupled with the one or more servers through a network 170, which could be a local area network (LAN), the Internet, or some other network. The ECG device may also be communicatively coupled with the one or more servers through the network, such as through a WiFi connection, or through one of the computing devices 166/168, or through another computing device—for example the ECG device could be connected through a wired connection to a personal computer (PC) or a tablet or the like which, in turn, is coupled with the one or more servers through the network.

The communicative coupling between computing devices 166/168 and the ECG device (and/or the one or more servers) allows the patient and clinician to interact with the ECG device, and/or to view data captured by the ECG device and/or to add information related to data captured or to be captured by the ECG device, using one or more user interfaces displayed on the displays 167/169. The communicative coupling between the ECG device and the one or more servers may allow software or firmware of the ECG device to be updated or edited and may allow data captured by the ECG device, or data input by the clinician or patient, to be stored in one or more data stores communicatively coupled with the one or more servers—and the one or more servers may themselves include or operate as one or more data stores. The one or more servers may include one or more database servers, one or more web servers, one or more application servers, and so forth. The one or more servers may run a software application (which may herein be referred to as a “software app” and which in implementations could be a mobile application) to be downloaded and run on the computing devices 166/168 to implement interactive user interfaces, and/or may run one or more website interfaces for users to access, view, edit, and/or download data and information captured by the ECG device or otherwise related to use of the ECG device. The servers may also perform processing operations on data captured by the ECG device, may facilitate user profiles and login credentials, and so forth, to facilitate use of the ECG devices and the user interfaces. Although the computing devices 166/168 are displayed as mobile phones, they could alternatively be tablets, laptops, PCs, or any other type of computing device.

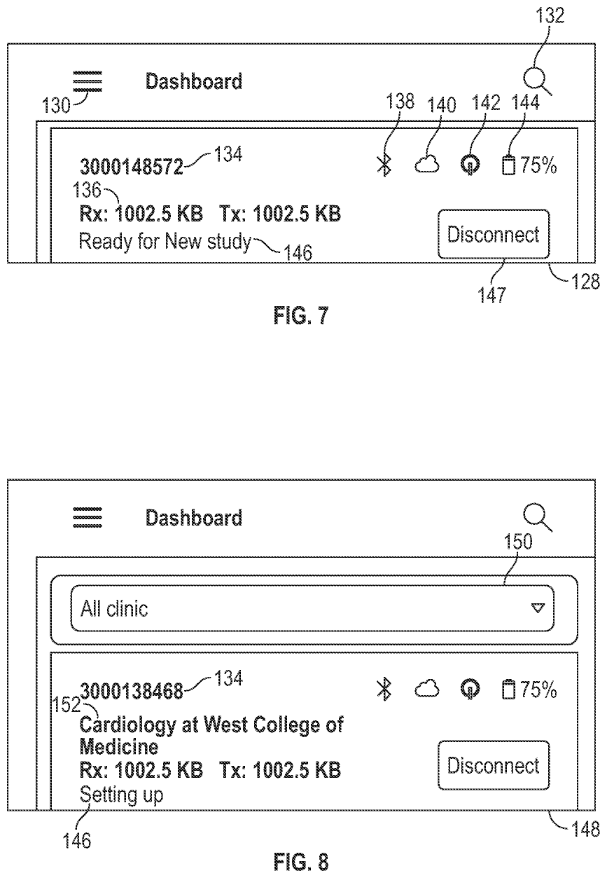

Referring now to FIGS. 7-11, a number of example user interfaces are shown. These are example user interfaces that may be shown to a clinician using the clinician computing device and/or to a patient using a patient computing device.

UI or user interface 128 of FIG. 7 is a clinician interface and includes a menu selector 130 which allows the user to navigate to other user interfaces or to logout or perform other operations and a search selector 132 which allows the user to search for a specific study or the like. A study designator 134 may include a numeric or alphanumeric indicator designating a specific study and may be input and/or edited by a clinician user or an administrative user. Server communication speed indicators 136 (Rx for receive speed and Tx for transmit speed) indicate communication speeds between the server and the ECG device and/or the computing device(s) 166/168. A recorder connection status indicator 138 indicates whether the ECG device is communicatively connected with the computing device in question, such as through BLUETOOTH or another wireless or wired connection. A server connection status indicator 140 indicates whether the ECG device is communicatively coupled to the one or more servers 164 (via one of the computing devices 166/168 or, in instances wherein the ECG device 100 has a dedicated cellular or other connection, directly through the Internet). An electrode connection status indicator 142 indicates whether all of the leads are coupled with electrodes and/or whether all the leads are electrically coupled with a patient through the electrodes. A battery level indicator 144 indicates a charge percentage of one or more batteries of the ECG device and whether the one or more batteries are charging (such as through a lightning bolt charging image). A study status indicator 146 indicates a status of the study (in this case the status is “Ready for New study”—this is the default status when the software app has successfully connected to the ECG device 100 and the one or more servers 164), which may be input by a clinician or administrator and/or automatically determined by the system. A disconnect selector 147 allows the computing device to disconnect the BLUETOOTH or other wireless connection to the ECG device.

To initiate a new study for a patient, an authorized clinic user may login to the BIOTRES Secure Server(s) using login credentials.

User interface 148 of FIG. 8 is a clinician interface. After the authorized clinician initiates the patient study at the BIOTRES Secure Server(s), the BIOTRES Gateway App clinician dashboard will display “Setting up.” This indicates that the study details are downloading from the BIOTRES Secure Server(s). The status indicator light 106 will change to a solid white color (waiting to start study) indicating the BIOTRES Recorder has received the study command. During this state the user may push the marking switch 104 and hold it for 5 seconds, to acknowledge and start the study. This interface includes a selector 150 which allows the clinician to filter studies shown on the user interface such as by all clinics, selected clinics, or using other criteria. Indicator 152 indicates a clinician or a clinic, or some other party or entity associated with a study, and may be input and/or edited by a clinician or administrator or other user.

After assuring a good BIOTRES Recorder patient hookup, and pressing the Marking Button for 5 seconds, the status indicator light 106 (which may herein be called “Status Indicator Light” or “Indicator Light” or “Status Light” with or without capitalization) will blink white for 30 seconds to acknowledge the start of the study and the sending of a baseline event. The status indicator light 106 should then change to slowly blinking green. This confirms that the ECG device 100 is hooked up to the patient correctly, and is engaged in a study. If the Status Indicator light fails to blink green and continues blinking white after 30 seconds, this could indicate that one or more electrodes are disconnected, or the study has been paused. If this occurs, the user may check the electrode connections to the Recorder and may check for correct placement on the patient's body, and/or replace the electrodes.

After the BIOTRES Recorder has downloaded the study details to the BIOTRES Dedicated Server(s), the clinician may abort the study by pressing and holding both the power switch 108 and Marking Button for 5 seconds. The Status Light will change from a solid white to solid green (indicating the BIOTRES Recorder is not engaged in a study), indicating that the study has been aborted. On the user interface(s) the study status will change from “Setting up” to “Ready for new study.”

It is not required that the patient use the BIOTRES Gateway App, but if the user does then the ECG device 100 may be coupled with the computing device, through the BIOTRES Gateway App, using the clinic credentials before allowing the patient to set up an account and use the BIOTRES Gateway App. If the patient is not using the BIOTRES Gateway App, the patient can be instructed to keep a log of symptoms when pressing the marking switch 104. After the study has been started, the ECG device 100 will record the patient ECG data. The status indicator light 106 will change to slow blinking green to indicate that study recording is in progress, with no pending transmission of the patient initiated manual events for the one or more servers 164.

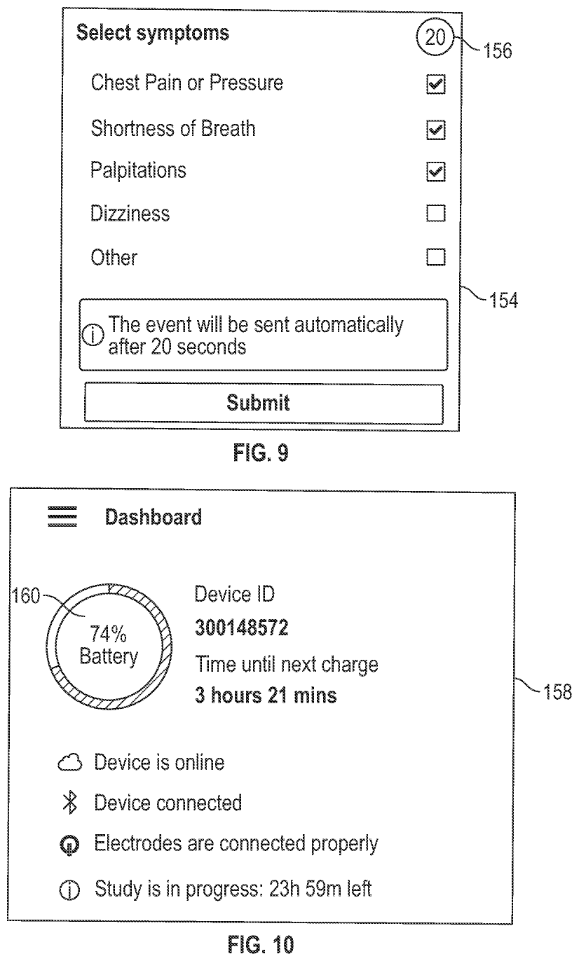

User interface 154 of FIG. 9 allows a user, such as a clinician or a patient (but in most cases a patient), to select one or more symptoms related to an event, by checking checkboxes. This interface may be presented to a patient, for example, on their computing device through the software app after pressing the marking switch 104, and may be used to associate selected symptoms with the event (the user may select more than one symptom per event). A countdown timer (timer) 156 counts down from a set time and, at the end of the countdown, automatically dismisses user interface 154. The user can submit the symptoms by selecting the SUBMIT selector in order to associate the symptoms with the event. In some implementations, when the user interface is automatically dismissed at the end of the countdown, the symptoms are not stored and/or associated with the event. In other implementations the symptoms are automatically stored and/or associated with the event at the end of the countdown even if the user does not select the SUBMIT selector, and the SUBMIT selector simply allows the user to dismiss user interface 154, and submit the symptoms to be stored and associated with the event, more quickly.

When the user presses marking switch 104 the status indicator light 106 may change to slow blinking white (slowly fading in and out) to acknowledge that a marked manual event is being recorded. If the user has downloaded the software app to a computing device, pressing the marking switch 104 may display user interface 154 to allow the patient to choose symptoms and press the Submit selector to record the symptoms.

FIG. 10 shows a clinician user interface 158 which includes details about the ECG device such as a battery status indicator (indicator) 160 indicating a charge level of one or more batteries, a device identification (ID), an amount of time the ECG device can operate until the next charge will be needed, and the aforementioned indicators 138, 140, 142, and 146. The interface indicates that a study is in progress and indicates how much time is left in the study.

During a study, the ECG device 100 may need to be disconnected by the patient for daily hygiene (such as a shower or bath). To do so, the patient turns the power off by pressing the power switch 108 for three seconds and then releasing it. The status light will change to fast blinking blue for three seconds and then the device will turn off. The user may then disconnect the ECG device 100 from the electrodes by gently pulling up on the leads one lead at a time. After the shower or bath or other need for removing the ECG device 100, the patient can then reconnect the ECG device 100 to the electrodes and then press and hold the power switch 108 for three seconds to turn the ECG device 100 on.

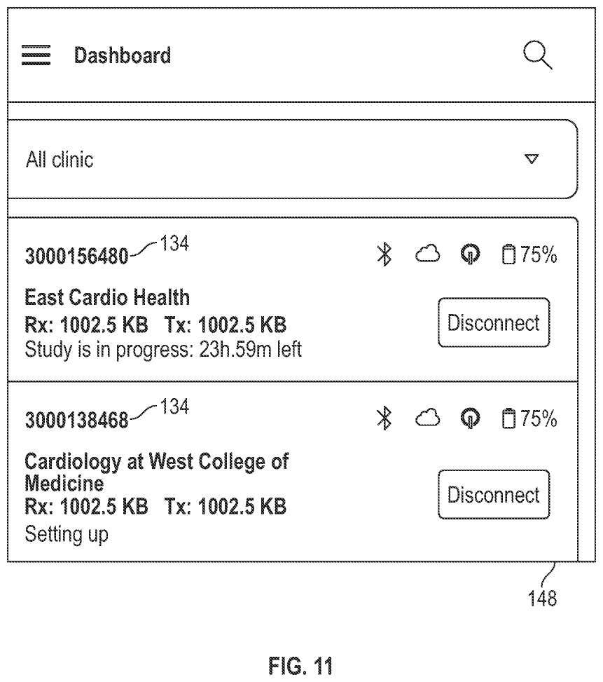

FIG. 11 shows a user interface 148 when multiple studies are displayed, each study area showing a study designator (designator) 134 and indicators 136, 138, 140, 142, 144, 146, 152, and disconnect selector 147. This is a dynamic user interface that will change based on what the user selects from the dropdown menu, what is occurring in each study (such as the time left in each study), the transmission and receipt speeds, the battery life, what studies are added or removed, and so forth. From this user interface a clinician can have a live view of the details being recorded/gathered by the ECG devices of multiple patients involved in multiple studies or otherwise being monitored.

In implementations other elements may be added, such as hardware and/or software elements for detecting/determining respiration, body temperature, motion/activity (such as accelerometer and/or gyroscopic sensors), blood oxygen, Peripheral Arterial Tonometry, Plethysmography, and any derivative biometrics (for example heart rate, atrial fibrillation, and HRV may be a derivative of electrocardiogram sensing/measurements, fall detection and sleep detection may be derivatives of motion/activity measurements, etc.). Associated user interfaces may display such measured or derived biometrics.

The ECG device may be considered a digital health wearable product that helps diagnose arrythmias faster, improving clinical outcomes and reducing risk. It addresses the challenges of current Holter monitors when detecting heart abnormalities in at-risk patients. It is the first of its kind, pushing the world of cardiac recording forward by addressing the issues of rechargeability (which enables long term recording) and connectivity (saving time for report analysis, leading to faster diagnoses and saving lives) which lead to inferior outcomes and delayed diagnosis. The ECG device 100 is a wireless 3-lead 3-channel Holter monitor and cardiac telemetry device and has a higher diagnostic yield and faster diagnosis. It is sleek and small in its design, allowing for greater patient comfort and reduced patient turnaround while providing superior support. In implementations the ECG device can record up to 48 hours without requiring a charge, making shorter 24-48 hour Holter studies very easy to complete. In implementations with charging the device can record up to 30 days of data. The ECG device can be worn in two different positions, and positions can be switched during longer studies for maximum patient comfort without compromising diagnostic yield. Patients can press the marking switch and report symptoms in an easy-to-use app so clinicians can be alerted to symptoms faster. Patient studies may be reviewed by highly trained certified cardiographic technicians (CCTs) and go through a double quality assurance (QA) process before being sent directly to clinicians' inbox within 72 hours of study completion, the fastest in the industry. The ECG device can capture data perpetually, not just temporarily.

The portable ECG device accordingly attaches to electrodes for a person to wear, and wireless connectivity to a software app (and to remote servers) allows for the recording of heart-related data. There is a clinician-side app with user interfaces and a patient-side app with user interfaces. The device is small, uniquely uses three electrodes to collect three ECG channels, and can record data perpetually (not just temporarily such as while the person is at the doctor's office). The patient can just wear it as they go about their day. The clinician can monitor the captured data remotely, from the clinician's office or through the clinician's computing device from any location, from time to time. This allows for the clinician to review much more data, over more time and over varied conditions/settings, to inform a diagnosis decision or the like.

Disclosed herein are ECG devices having small form factors but which use standard electrodes. For example, ECG devices disclosed herein have dimensions equal to or smaller than 116.5 mm by 85.5 mm and which use standard electrodes having a contact point (male snaps or other contact point) of 4 mm by 4 mm or smaller, or 3.9 mm diameter or smaller, the standard electrodes having overall dimensions equal to or smaller than 4 inches by 4 inches (for rectangular electrodes) or having diameters of 4 inches or smaller for circular electrodes. The phrase “standard electrode” is defined herein as any electrode having a contact point (male snaps or other contact point) of 4 mm by 4 mm or smaller, or 3.9 mm diameter or smaller, and having overall dimensions equal to or smaller than 4 inches by 4 inches (for rectangular electrodes) or having diameters of 4 inches or smaller for circular electrodes.

In implementations, to ensure proper studies the clinician may verify the ECG signal quality upon securing it to the patient by looking at the patient's baseline. A single channel may in implementations be sufficient for proper diagnosis, but three channels are ideal in implementations. In some cases all channels may not appear to do lack of electrode connectivity or the like, but in implementations it may take up to five minutes for electrodes to warm up and display a proper signal, and this may vary from individual to individual. The clinician may ensure the ECG device is connected by checking once a day on existing patients for artifacts, battery, or missing data, and can follow up with the patient as necessary. The clinician can explain to the patient how to use the ECG device and that it should be charged every 48 hours (or on some other time frame), and to only use a supplied alternating current (AC) wall charger to do so. The clinician can also ensure the patient's name, address, phone number, study type, etc. are entered correctly. In some cases where connectivity is bad or elements are temporarily disconnected, there may be pauses in being able to view data, though the data may be able to be viewed later once connectivity is re-established.

In implementations the ECG device 100 is an ECG recorder only, does not include ECG analysis or alarms, and is not a real time ECG monitor. In implementations ECG device 100 is a small, lightweight, ambulatory, reusable 3 channel ECG recorder that is placed on the center of the chest.

While the ECG devices are BLUETOOTH based in some implementations, in other implementations they may have integrated cellular plans such that they are wirelessly connected to a network and/or through cell towers and the Internet for communication with remote servers and the like and will not rely on a user's computing device for connectivity therewith. In such implementations, however, the user may still need a computing device to access some user interfaces associated with software applications.

The patient may push the marking switch 104 when the patient is feeling symptoms such as palpitations, shortness of breath, dizziness, light-headedness, pre-syncope, syncope, fatigue, chest pain, anxiety, etc. The marking switch 104 in implementations houses an LED status indicator light 106 in its center, though the status indicator light 106 may be internal (and illuminating therethrough) or external to marking switch 104, and may have a variety of different configurations, and may in some cases include multiple LEDs or other lights.

The status indicator light 106 may provide status indications when: ECG device 100 is powering on; electrodes are disconnected; the battery is low; the ECG device 100 is charging or is fully charged; the ECG device 100 has encountered an internal error; the patient has pressed the marking switch; a study has or is not engaged, started, running, paused, or completed; and so forth.

In implementations the ECG device 100 attaches to the patient's chest through three standard commercially available ECG electrodes (in implementations not included with the system but supplied by a clinic or a monitoring center). In implementations ECG device 100 includes a BLUETOOTH modem or chip or communication elements (or other wireless communication elements) to communicate with one or more secure servers (which may herein be called “BIOTRES Configured Secure Server(s)” or “BIOTRES Secure Server(s)” or “BIOTRES Server(s)” with or without capitalization) through one or more software applications (which may be herein called the “BIOTRES Gateway App” or “the software app” with or without capitalization). Additionally, the ECG device 100 may herein be called the “BIOTRES Recorder” or “Recorder” with or without capitalization. Integrated memory of the ECG device 100 may in implementations store up to 30 days of ECG data (though in some cases more or less than 30 days of data). This data can be uploaded by the clinician to the BIOTRES Configured Secure Server(s).

A dedicated AC wall charger may be used to charge the BIOTRES Recorder daily when disconnected from the patient.

In implementations the ECG recorded data is transferred from the ECG device 100 to the BIOTRES Gateway App (downloaded from the appropriate app store for iOS or ANDROID, as examples) via a BLUETOOTH connection and then subsequently to the BIOTRES Configured Secure Server(s). Clinicians can access the BIOTRES Gateway App using their privileged login credentials to connect BIOTRES Recorders to the BIOTRES Configured Secure Server(s) so that patient studies may be started and completed. The BIOTRES Gateway App can be accessed by the patient once registered (e.g., using nonprivileged login credentials) and the patient can use the app to monitor the status indicators of the BIOTRES Recorder and to enter symptoms if the marking switch 104 (also called herein the “Event Marking Button” or “Marking Button” with or without capitalization) is pressed on the ECG device 100. The ECG recorded data from the BIOTRES Recorder can be transferred, by the clinician, from the BIOTRES Configured Secure Server(s) when the ECG device 100 is connected to the BIOTRES Gateway App at the end of a patient study.

The BIOTRES Configured Secure Server(s) may be Secure Shell File Transfer Protocol (SFTP) server(s) configured to work with ECG system 162 and may enable the clinician, through secured login, to command the BIOTRES Recorder (e.g. initiate ECG data collection) and to access uploaded MIT16 ECG data recordings when the ECG data collection is complete (MIT16 is a MASSACHUSETTS INSTITUTE OF TECHNOLOGY 16-bit format). In implementations ECG device 100 does not provide the patient or clinician with diagnostic or interpretive statements based on the ECG data. Diagnosis and interpretation of recorded ECG data may instead be dependent upon the medical advice and guidance provided by a physician or trained healthcare professional. The ECG device 100 in implementations is for home use, though clinicians could use it in a hospital setting if desired. Clinical judgment and experience may be used to check and interpret the data.

In implementations the ECG recordings can be viewed through a software tool by qualified medical personnel called BIOFLUX SOFTWARE II (K201040). This ECG viewer software may display ECG records in a browser on a computing device and may provide tools for trained clinicians to annotate, store, retrieve, and report ECG recordings. It may be utilized by manually opening ECG files or through an application programming interface (API) that allows for third parties to integrate their devices and directly push ECG files into the viewer. In some implementations ECG recordings can be processed or viewed through VASOmedical ARCS (or other verified third party ECG analysis tools, such as using APIs) and analyzed by qualified healthcare professionals.

In implementations ECG device 100 may be indicated for use on patients who may be asymptomatic or who suffer from transient symptoms such as palpitations, shortness of breath, dizziness, light-headedness, pre-syncope, syncope, fatigue, chest pain and/or anxiety and who may require or benefit from cardiac recording on a continuous basis for up to 30 days (or for some other time frame shorter or longer). In implementations the ECG device 100 may be used by patients and by healthcare professionals such as cardiologists, physicians, general practitioners, nurses, cardiac/ECG technicians, monitoring service technicians, diagnostics testing technicians, and other healthcare professionals.

In implementations clinician and/or patient uses may enable all access controls on the computing device(s) running the BIOTRES Gateway App to protect from unauthorized use such as by incorporating password and/or biometric protection/control (e.g., not allowing use of the app without entering the computing device's password or providing biometric identification).

As discussed, the marking switch 104 is used to trigger patient-initiated events, and may have a built-in or coupled/attached status indicator light 106 to display a status of the ECG device 100. The status indicator light 106 may include a plurality of separate lights and/or may be otherwise capable of displaying various colors and light patterns. By non-limiting examples, the following colors and display patterns could have the following indications/meanings:
1. Solid green: ECG device 100 is powered on and not engaged in a study.
2. Solid white: ECG device 100 has received a “Start Study” commend, the patient or clinician can acknowledge this by depressing the marking switch 104 for 5 seconds.
3. Slow blinking green: ECG device 100 is engaged in a study.
4. Blinking blue: low battery.
5. Slow blinking blue: battery is charging.
6. Blinking yellow: ECG device 100 has encountered internal error(s).
7. Solid blue: battery is fully charged.
8. Blinking white: electrodes disconnected or study paused.
9. Blinking green: study completed and being uploaded.
10. Fast blinking blue for 2 seconds: ECG device 100 is powering on.
11. Slow blinking white: user-marked manual event is accepted and processed.

In implementations a user may turn on the BIOTRES Recorder by pressing the power switch 108. In some cases the status indicator light 106 will flash for 3 seconds in a blue color and then stay solid green, indicating that the recorder is powered on and is not engaged in a study.

ECG device 100 can be charged after being disconnected from the patient electrodes. In implementations the electrodes may be left on the patient and the ECG device 100 can be reconnected to the same electrodes after charging, though in other implementations the electrodes may be replaced before reattaching ECG device 100.

In implementations the ECG recorded data is transferred to the BIOTRES Gateway App (e.g., to the computing device using the app) via a BLUETOOTH or other wireless connection and then subsequently to the BIOTRES Configured Secure Server(s). Clinicians can use the BIOTRES Gateway App using their login credentials to connect BIOTRES Recorders to the BIOTRES Configured Secure Server(s) so that patient studies may be started and completed. The BIOTRES Gateway App can be used by the patient once registered (nonprivileged login credentials) and the patient can use the App to monitor the status indicators of the BIOTRES Recorder and to enter symptoms if the Event Marking Button is pressed on the Recorder. The ECG recorded data from the BIOTRES Recorder can be transferred, by the clinician, from the BIOTRES Configured Secure Server(s) when the ECG device 100 is connected to the BIOTRES Gateway App at the end of a patient study (for example the clinician may transfer the data from the one or more servers 164 to analyze the results and/or provide a diagnosis).

After downloading/installing the software app, the user may be brought to a login page allowing them to sign in (or sign up) as a patient or clinician.

In implementations ECG device 100 may be used with conventional lead electrodes supplied to a patient by a physician or monitoring center. 3M RED DOT SOFT CLOTH MONITORING MODEL 2238 electrodes may be used, as a non-limiting example, or any other Food and Drug Administration (FDA) cleared snap/lead electrodes. Some electrodes may be worn for up to 3 days, then should be discarded and replaced. Conventional skin preparation techniques, such as shaving hair and cleaning skin, may facilitate a good electrode connection. In implementations the electrodes may be snapped or connected with ECG device 100, and then backing removed from each electrode, prior to the electrodes being placed on the user's skin to adhere thereto. In some cases the electrodes are placed over bone structures, as artifact and noise may result from placement over large muscles or fatty tissue.

When using the centers position of FIG. 5 (for males and females), the white (RA) lead may be placed in an upper-right portion of the chest, two to three inches below the collarbone. The red (LL) lead may be placed in the center of the chest. The black (LA) lead may be placed on an upper-left portion of the chest, two to three inches below the collarbone.

When using the alternative electrode placement of FIG. 6, the white (RA) electrode may be placed two to three inches below the left collarbone and sternum junction. The red (LL) electrode may be centered between the RA and LA electrodes. The black (LA) electrode may be placed two to three inches below the left collarbone.

As indicated the BIOTRES Gateway App may be downloaded and installed by both the clinician and the patient on their respective computing devices. Both can communicatively couple their computing devices with the respective ECG device 100 to set up a study and so that data may be uploaded at the end of a study. The BIOTRES Gateway App in implementations provides a secured communication bridge between the ECG device 100 and the one or more servers 164 (BIOTRES Configured Secure Server(s)). The clinician version of the software app may be used at the clinic during patient hook up to the ECG device 100 for an ECG Holter study.

In some cases, after charging for two hours the ECG device 100 can continuously operate for up to 48 hours. To charge the device, the user first powers it off, detaches the leads from the electrodes, and plugs a charger into the ECG device 100 and into a wall outlet. The status indicator light 106 will slowly blink blue during charging and will turn solid blue when fully charged. The user may then reattach the leads to the electrodes, power on, and the status light will change to slow blinking green to indicate that the study/recording has resumed.

When a study is completed the study status indicator 146 on the clinician user interface(s) may show “Study completed” and the status indicator light 106 may change to continuous blinking green. The data may then automatically begin uploading to the one or more servers 164 and the study status indicator 146 may change to “Uploading study data.” The study data may be uploaded automatically to the one or more servers 164 once the study is complete. The ECG device 100 will be ready for a new study after the data for the completed study is uploaded to the one or more servers 164 and erased locally from ECG device 100 (and/or from the computing device(s)). The status indicator light 106 will change to solid green indicating the ECG device 100 is no longer engaged in a study, and the study status indicator 146 will show “Ready for a new study.”

The status indicator light 106 will change to blinking white if one or more electrodes are disconnected from the leads. In the software app the electrode connection status indicator 142 will change from green to gray and may display “One or more electrodes are disconnected.” If this occurs the patient or clinician can check the electrode connections/snaps.

The status indicator light 106 will blink blue when the battery charge is low. In some cases the battery should be charged for a minimum of an hour per day. The patient can monitor battery level through the BIOTRES Gateway App such as using any of the interfaces shown in the drawings (which, although some are discussed as being clinician interfaces, may also be visible to the patient through the software app on the patient's computing device—or redacted/simplified versions may be shown to the patient).

The status indicator light 106 will change to continuous blinking yellow if an internal error is detected. The user can try turning off the ECG device 100 and powering it on again to see if this resolves the issue.

The ECG device 100 may be cleaned between uses such as with alcohol and/or other solutions so that it may be used on different patients and/or to otherwise keep it clean.

In implementations the ECG device 100 has collects three ECG channels using three electrodes.

When setting up a new study, from a dashboard interface such as that of FIG. 11 the clinician may scroll to a device from the available list of study designators 134 (which study designators may, in some cases, be the same as a serial number on a given ECG device 100) (the device may show up on user interface 148 as being available if within wireless range and detected but may not have Rx/Tx values or have any indicated status). A popup may ask whether the clinician wishes to connect the ECG device 100 and, upon selecting yes, a “Connecting” popup may appear, followed by a “Success” popup upon pairing/connecting or a “Failed, please try again” popup on failing to pair/connect. Upon successful connection the user will be directed back to the dashboard screen, such as that of FIG. 11, where the newly connected ECG device 100 may be shown along with Rx/Tx values and the status of “Setting up.”

In implementations, when the recorder connection status indicator 138 is green, the ECG device 100 is connected to the software app and/or computing device via BLUETOOTH or another wireless coupling. When it is gray the ECG device 100 is disconnected from the software app/computing device. This can happen if the ECG device 100 is more than 10 meters away from the computing device, the ECG device 100 has been powered off, or BLUETOOTH or another wireless mechanism has been disabled on the computing device.

When server connection status indicator 140 is green the ECG device 100 is connected or communicatively coupled to the one or more servers 164 either directly through the Internet (if it has a standalone cellular or other connection to the Internet) or indirectly through one of the computing devices 166/168. When it is gray the ECG device 100 is disconnected from the one or more servers 164.

When the electrode connection status indicator 142 is green, all of the ECG device's electrodes are connected. When it is gray one or more of the electrodes are disconnected.

The following variations for study status indicator 146 may have the following meanings:

Ready for New Study: no study is currently being run, and data from any previous study has been erased locally from the ECG device 100 and/or computing device(s).

Setting Up: the ECG device 100 has received a study initiation request from the one or more servers 164 and is waiting for the user to start the study.

Study in Progress: the ECG device 100 is engaged in a study and recording ECG data.

Study Paused: the ECG device 100 is engaged in a study but is not presently recording ECG data—this occurs when the user removes the ECG device 100 (e.g., to charge or bathe). This status may also appear as orange (and the battery level indicator 144 may appear orange while charging), while at all other times the study status indicator 146 may appear as blue, such as to more easily catch the user's attention to remind the user to reconnect the ECG device 100 after bathing or charging.

Study Completed: the study has run its course, or was stopped by a clinic through the one or more servers 164.

Uploading Study Data: the ECG device 100 is uploading study data to the one or more servers 164.

Study Uploaded: the upload of study data to the one or more servers 164 has completed.

When the ECG device 100 is plugged into a charger, battery level indicator 144 changes to show a lightning bolt within the battery image (to indicate charging) and the battery level indicator 144 changes to orange and displays the estimated charge percentage remaining. When ECG device 100 is not plugged into the charger the battery level indicator 144 is green and displays the battery image without a lightning bolt therein and displays the estimated charge percentage remaining.

A user (clinician or patient) may sign out of the BIOTRES Gateway App and all connected ECG devices 100 will be automatically disconnected from the corresponding computing device. An ECG device 100 would then need to be reselected upon logging back in in order to view the recorder data and study status again (in other words, the recording may continue, but the recorded data and other details may not be visible to the user until the user has logged back in and reconnected the computing device with the ECG device 100). A user can sign out by tapping menu selector 130 and then tapping a “Sign Out” selector.

In implementations the ECG system 162 and/or software app may be configured to do the following: provide a push notification to the user if the ECG device 100 failed to power on; implement a hardware watchdog timer to avoid system latch-ups; send health check status of the ECG device 100 to the one or more servers 164 at predetermined frequencies/intervals if the ECG device 100 is connected to a computing device that is online; notify the patient or user via push notification when the battery charge goes below a predetermined threshold; perform daily cyclic redundancy checks (CRCs) to detect bit flips due to environmental factors (and alert the user via push notification upon CRC failure); notify the user via push notification upon the battery being fully recharged; notify the user via push notification when a manual event has been recorded/logged and/or associated with one or more symptoms and/or if logging and/or symptom association fails; notify the user via push notification when a study (or study time) completes and/or when ECG data is uploaded; notify the user via push notification if the devices logs or experiences an error; and automatic powering down when battery charge drops below a predetermined threshold. In implementations some predetermined thresholds or values may be adjustable by the clinician or patient and/or some push notifications may be paused or turned off. Any push notifications discussed herein may be done by a popup interface, or by a notification indicator on any user interface of the user's computing device (which in some cases a user may select to see the notification expanded and/or to show details of the notification using the software app).

In some cases the clinician could, through one or more user interfaces, change a study from a Holter study to a mobile cardiac telemetry (MCT) study remotely from the ECG device 100 and without having the patient come into the clinic/office. The clinician may also in implementations start and stop a study remotely from the ECG device 100 and without having the patient come into the clinic/office.

In implementations the ECG device does not include ECG analysis, an ECG viewer or display, or alarms and is not a real time ECG monitor. In other implementations this may change. The ECG device may be modified to include built-in algorithms for ECG analysis, an ECG viewer or display, alarms, and/or may conduct (and allow viewing of) real time ECG monitoring. The ECG device 100 may include and may use one or more algorithms stored therein/thereon and/or may interface with the one or more servers 164 which run one or more algorithms remotely to accomplish the functions disclosed herein.

In implementations the ECG recorded data may be transferred to the BIOTRES Gateway App (i.e., to the computing device using the app) via a BLUETOOTH connection. In other implementations this may change, and the data may be transferred via a software app or directly to remote servers by a new version of the ECG device which includes integrated cellular communication technology. In implementations clinicians can use the BIOTRES Gateway Application using their privileged login credentials to connect ECG devices (BIOTRES Recorders) to the BIOTRES Configured Secure Server(s) so that patient studies may be started and completed. In implementations studies may be initiated via the software application and/or through a web/cloud portal.

In implementations ECG device 100 stores recorded data until uploaded to the one or more servers 164, at which point it is erased/removed from the ECG device 100. The data may be compressed while stored on ECG device 100. The ECG device 100 may be configured to check the integrity of the recorded ECG data before or during upload to the one or more servers 164.

In implementations the clinician may be notified (such as via push notification) if a patient's lead(s) are disconnected for 30 seconds or more and/or if the study is paused (such as automatically when the user powers down the ECG device 100 and begins charging it or removes it for bathing). If the lead(s) are not reconnected and/or the study automatically restarted within a reasonable amount of time, the patient may be contacted by the clinician and/or an administrator and/or other party/service to check the reason/status.

In implementations, after the ECG device 100 is powered on, it will return to the mode it was in when powered down (for example automatically returning to ECG recording).

The one or more servers 164 may record and store timestamps and indications such as when a study starts, when a study is aborted, when a study finishes, etc. In implementations the uploading of ECG recorded data is initiated by ECG device 100 after a study completes or when it enters another predetermined mode, such as during charging, or at regular predetermined intervals—though if battery charge is low, such as around 3%, the device may wait to upload the data until the battery is more fully charged or is charging so that the upload is not interrupted by the ECG device 100 powering down. In implementations the ECG recorded data for any specific study is not erased (even if some of it has been uploaded) until the study fully completes. In other implementations any ECG recorded data already uploaded from ECG device 100 to the one or more servers 164 may be erased from ECG device 100 even if the study has not yet completed.

The ECG device 100 may keep logs of all software events, commands, signals/messages, errors, etc. such as for later troubleshooting and analysis if needed. For example, the logs may include power-up diagnostics for hardware reliability checks, communication errors between the ECG device 100 and/or computing device and the one or more servers 164, ECG data upload errors to the computing device and/or in turn to the one or more servers 164, detectable hardware failures, faster than normal battery depletion, etc. The ECG device 100 software may maintain battery measurement trending data in order to assess remaining usage (expressed in terms of remaining time and/or remaining charge percentage) before the next needed charge.

The ECG system 162 and/or ECG device 100 may provide/initiate push notifications to the user when a battery is low and/or critical and needs charging and/or when it is charging, when a lead is disconnected and/or reconnected, when the ECG device 100 is powered on or powered down (or when a power down has been requested), when ECG device 100 experiences any error, when power on initialization fails, when data upload to the one or more servers 164 fails, etc. Recorded data, including Protected Health Information (PHI) may be encrypted prior to uploading from the ECG device 100 to a wireless (e.g., BLUETOOTH) connected computing device and before uploading from the computing device to the one or more servers 164. Local PHI may be deleted from the ECG device 100 and/or from a computing device running the software app (though stored on the one or more servers 164) after a study starts. Wireless communications between the ECG device 100 and a computing device (such as via BLUETOOTH) may be encrypted.

Various example operating system and software details for ECG device 100 will now be described. In implementations ECG device 100 includes an in-house basic real-time operating system (RTOS) optimized for its hardware environment. This RTOS provides fundamental services to: facilitate communication between objects and modules; manage interrupts; control software execution; and provide general purpose timer services. In this sense, these services play an underlying role in fulfillment of all software requirements.

Individual objects in this subsystem are facilitate software function and thus will be described below. These include an Event Manager, an Interrupt Manager, an Execution Manager, a Timer Manager, and a Static Scheduler.

The Event Manager implements an event-messaging system for communication between software objects. Events may be queued and dispatched to any known event handler. Objects are responsible for registering their event handlers with the Event Manager during initialization. The Event Manager provides additional support for error checking and it maintains event buffer integrity.

The Interrupt Manager maintains the interrupt control masks and vector tables of the ECG device 100 and provides handlers for unused interrupts. Objects are responsible for registering their interrupt service routines (ISRs) when necessary, and for returning interrupt vectors to the unused interrupt handler when no longer needed.

The Execution Manager provides an executive loop from which events are dispatched. It also provides a non-markable interrupt (NMI) handler, controls and assesses resets due to software or hardware errors, maintains mode-appropriate code and data protection in random access memory (RAM), and provides an efficient mechanism for critical section (uninterrupted execution) protection.

The Timer Manager provides services to allocate, set, and reset multiple general purpose hardware timers. An ISR is provided for each timer. When a timer “rings,” its ISR dispatches an event to the requesting object. The Timer Manager also provides routines to set and read the real-time clock of the ECG device 100.

The Static Scheduler provides services to initiate activities at a certain time each day or each number of days. For example: measure the rechargeable battery voltage at 7 AM each morning; and/or check electrode impedances once per week.

Operating Mode Control modules manage transitions between Bootup Mode, Non-Recording (low power) Mode, and Recording Mode. Mode monitors install event handlers relevant to their modes and they dispatch initialization control to each relevant object. A typical path from Bootup Mode to Recording Mode is described below.

The Application Loader completes ECG device 100 initialization upon power up or reset. During this process, the flash CRC Verifier object confirms integrity of each memory segment. Flash corruption is repaired by having the Memory Copier object overwrite any bad segments with a backup copy stored in an external or internal flash chip or other memory module. Once all segments have been loaded and verified, the Application Loader passes control to the Non-Recording Mode Monitor. The Non-Recording Mode Monitor then finalizes all Bootup Mode objects and initializes all Non-Recording Mode objects. If progression to Recording Mode is later requested, the Recording Mode Monitor similarly finalizes all Non-Recording Mode objects and initializes all Recording Mode objects.

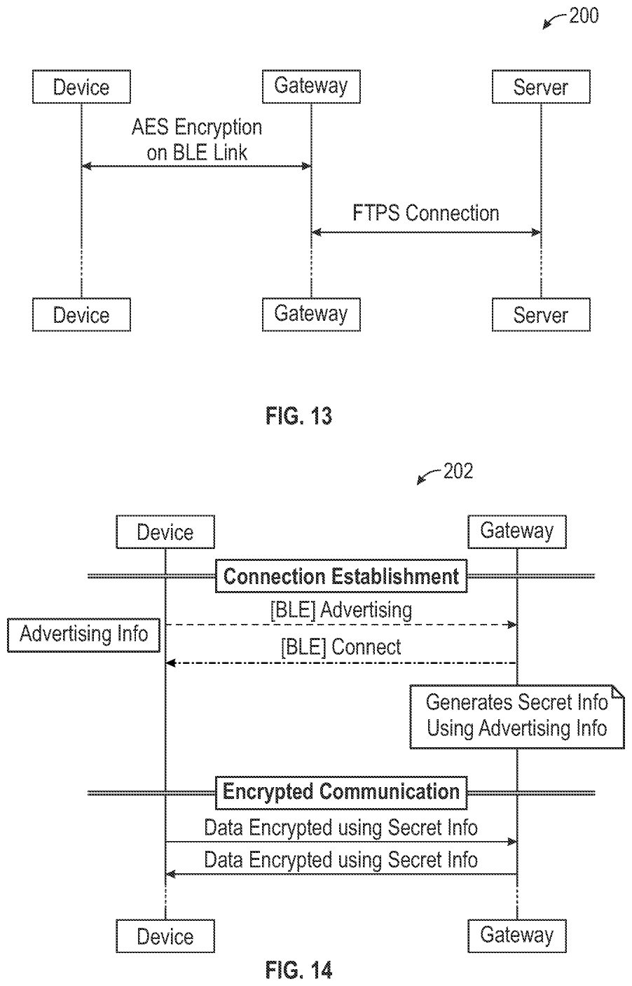

A Wireless Communication Manager object is responsible for communication with the remote one or more servers 164 via a Bluetooth gateway. The Wireless Communication Manager object receives the command requests from the remote one or more servers 164. This object is responsible for responding to the remote server as per the requested commands. It is also responsible for communications timeout interrupt, maintaining download and upload data stream status, rejecting inappropriate or uninterpretable requests, updating Programmable Parameters, and requesting the transition to Recording Mode upon configuration update. These elements are representatively illustrated by diagram 200 of FIG. 13 wherein “Device” represents ECG device 100, Advanced Encryption Standard (AES) encryption is used for BLUETOOTH Low Energy (BLE) communications between the ECG device 100 and the software application (Gateway) of the user's computing device, and a File Transfer Protocol Secure (FTPS) standard is used for communications between the user's computing device and the one or more servers 164. The one or more servers 164 may, at any given time, be involved in communications with any number of software applications (Gateways, on any number of computing devices) and any given Gateway may in turn be involved in communications with any number of ECG devices 100, and thus multiple ECG devices 100, Gateways, and Servers are illustrated in FIG. 13.

With regards to device-level security, referring now to diagram 202 of FIG. 14, in implementations communication between ECG device 100 and the software application (BLUETOOTH gateway) of the user's computing device are protected by encryption on the BLE link which involves a 256-bit Secure Hash Algorithm (SHA-256) and 128-bit or 256-bit Advanced Encryption Standard (AES-128 or AES-256) symmetric encryption algorithm (Counter or CTR mode). The initialization of BLE link encryption is described as follows:
1. The Bluetooth Gateway scans for nearby devices while the BIOTRES device introduces itself via
2. The Bluetooth Gateway (e.g., software application on the user computing device) scans for nearby ECG devices 100 while the nearby ECG device 100 introduces itself via BLE advertising.
3. Upon establishing the connection to the ECG device 100, the Bluetooth Gateway generates secret information by hashing the advertising info of the nearby ECG device 100 (using SHA-256).
4. From that point onward, packets traveling between the nearby ECG device 100 and Bluetooth Gateway in either direction are encrypted by an AES-128 or AES-256 encryption algorithm.

In implementations an ECG recording subsystem of ECG device 100 implements an allocation scheme to store the compressed electrocardiogram (ECG) data on a Secure Digital High Capacity (SDHC) memory card, using a File Allocation Table (FAT) file system. This may be designed in a way that any chunk of one-second ECG data can be referenced via a unique epoch timestamp.

In cooperation with an ECG Shuttle analog front-end (AFE) hardware and an ECG Buffer functional module, the ECG Recorder object in this subsystem manages triggering, initiation, and completion of patient commanded events. Data structures may be maintained to enable traversal of a list of valid, commanded event records categorized by trigger time.

In implementations the Electrocardiogram (ECG) Recording subsystem operates in close cooperation with the Wireless Communication Manager object. The remote one or more servers 164 may request to read recorded ECG data by providing a specific time range that covers the ECG data segment it wants to get.

Objects in this subsystem are also responsible for maintaining diagnostic, ECG, and scratchpad information across a device reset in case of unexpected ECG device 100 failures.

In implementations a Measurement subsystem samples a range of system inputs, including rechargeable battery voltage and lead impedance. Sampling may be performed in close cooperation with the Measurement analog-to-digital (ADC) hardware. Measurement requests may be issued via the one or more servers 164. Upon completion of a measurement, the Measurement Manager object may send a result message to the requesting object. The most recent measurements may also be cached in RAM of the ECG device 100 by the Measurement Manager object for later retrieval.

A Battery Voltage Trend object may implement a periodic check of the battery voltage. A transition to Non-Recording (low power) Mode may be requested when the battery voltage falls below a factory-calibrated “Turn Me Off” threshold.

A Pending Battery Measurement object may remember when a measurement must be delayed to avoid contention with other activities that may affect the measured voltage.

A Code CRC Check object may be periodically asked by the Static Scheduler object to check for and repair memory corruption.

In implementations each hardware block implemented on the ECG device 100 may be accessed through an interface functional module. At the minimum, these interfaces may provide accessor macros and/or functions to read from and write to a block's hardware registers. Interfaces can also emulate, in software, functions that may migrate to future hardware implementations.

In implementations Hardware Interfaces of ECG device 100 are used throughout the software and may be generally involved in fulfillment of some or all software requirements.

The data structure of ECG device 100 in implementations includes programmable parameters, module working data, system-wide shared data, write protected data, device lifetime data, and hardware calibration data (TRIMS data structure).

The working set of programmable parameters of ECG device 100 may dictate activity recording operations. The programmable parameters may be write-protected and may be backed up to nonvolatile memory. New parameters may be downloaded to the device as a temporary set. Once verified as uncorrupted with all critical parameters within acceptable limits, the temporary set may replace the present working set in a protected critical section.

With regards to module working data, each software object or functional module may allocate working data in this data structure. Working data in implementations are not write-protected. In general, objects may avoid direct access to other objects' working data.

System-wide shared data may generally be write-protected. Such data may hold important basic data used by the application to track external RAM allocation and information needed at operating mode changes.

Write protected data may be backed up to nonvolatile memory and write-protected. It may hold object-specific data (battery status, for example) that ideally would persist across operating mode transitions and across ECG device 100 resets.

Device lifetime data may generally be write-protected. It may hold status and counter information that the application maintains over the life of the ECG device 100. In implementations these data cannot be directly modified by the remote one or more servers 164.

Hardware calibration data may be write-protected and backed up to nonvolatile memory. It may hold unit-specific values defined during calibration in manufacturing to “trim” hardware circuits of ECG device 100 for optimal performance.

Further example details of the software app(s) of ECG system 162 will now be given. These are only examples and other implementations may include other details.

A software application (BIOTRES Gateway App) in implementations is a software utility used to manage data communication between a BLUETOOTH module/element of ECG device 100 and one or more servers 164. In implementations the app installs on commercially available, dedicated, mobile devices based on the latest iOS or ANDROID operating systems, though in other implementations it may be installed on other computing devices such as laptops, tablets, smart watches, smart glasses, desktop computers, and so forth running the WINDOWS, LINUX, MAC OS, or any other operating system. In implementations the one or more servers 164 host and/or provide the software app for download, update, and so forth.

Under normal operation, when a connected/paired computing device is within wireless BLUETOOTH range of an ECG device 100, the software app connects to the ECG device 100 over BLUETOOTH and establishes a secured data exchange with the one or more servers 164 using the computing device's WiFi or cellular connection. In implementations the software app is required to relay the ECG device 100 setup parameters from the one or more servers 164 and to relay notifications and the ECG data from the ECG device 100 to the server. The ECG device 100 may buffer or delay notifications and ECG data upload to the one or more servers 164 if the software app is not available due to any interruption in wireless communication.

In addition to data communication relay between the ECG device 100 and the one or more servers 164, one or more user interfaces of the software app display various operational statuses of the connected ECG device 100. The displayed device statuses and/or push/popup notifications may include, for example: remaining battery level, electrode connection status or reliability, server connectivity, and other items. In implementations the software app may be configured to run in the background on a computing device. It may be configured to initiate scanning of nearby ECG devices 100 and to connect therewith over BLUETOOTH LE. The software app may have permissions to access the computing device's location, storage, and to access the Internet. It may be configured to connect with more than one ECG device 100 at a time and may automatically reconnect to previously-connected ECG devices 100 when within range.

Upon receiving data from connected ECG devices 100, the software app may be configured to perform a cyclic redundancy check (CRC) to ensure data integrity. The app may also be configured to receive electrode connection status, battery status, study status (e.g., Ready for New Study, Setting Up, Study is in Progress, Study Paused, Study Completed, Uploading Study Data, and/or Study Uploaded) of connected ECG devices 100. The software app may authenticate user credentials (e.g., email and password) with the one or more servers 164. The software app may support distinct user roles for clinicians and patients, as described to some extent above, and accordingly may utilize different user interfaces, request different information, and so forth for the different user types.

In implementations the software app may establish a Transmission Control Protocol (TCP) connection with the one or more servers 164 and may automatically retry establishing the TCP connection if it is disconnected. The software app may relay messages from the one or more servers 164 to connected ECG devices 100 and may forward notifications and data from connected ECG devices 100 to the one or more servers 164. In implementations the software app does not (and/or may not) access PHI data exchanged between connected ECG devices 100 and the one or more servers 164. Communication between the software app and its connected ECG devices 100 may be encrypted using AES-128 or AES-256 encryption, and communication between the software app and the one or more servers 164 may be encrypted using Transport Layer Security (TLS) technology.

The software app may be configured to display, using its one or more user interfaces, a list of ECG devices 100 connected with it along with, for each connected ECG device 100: a BLUETOOTH connection status; an electrode connection status; a battery status (including battery level, whether the battery is charging/discharging, and if charging then estimated time to full charge). The software app's user interface(s) may display a connection status between the software app and the one or more servers 164 and/or between the connected ECG devices 100 and the one or more servers. If a previously-connected ECG device 100 is not connected to the software app, its battery status, electrode connection status, study status, and server connection status may be displayed as “Not Available.” In implementations only a clinician user may add and remove ECG devices 100. Once added, though, the patient user may connect with an added ECG device 100 using the software application on the patient's computing device, such as in the clinician's office during a visit in which the clinician explains the use of ECG device 100 to the patient. In implementations, patient users may only see devices/studies which are in statuses of (or statuses which indicate studies which are) started, paused, resumed, completed, uploading, and uploaded.

Further example software elements of the software application will now be discussed. These examples are intended to be an overview and may not discuss all communication paths and software object interdependencies, nor is every software object or use case variation described, as such is not needed by the practitioner of ordinary skill in the art to implement the software application and the functions described herein.

In implementations an ALAMOFIRE networking library written in SWIFT is used to build network functions which are used for the software application to establish TCP network connection with the one or more servers 164, exchange data, and secure the communication over the connection with TLS technology. TERMINAL I/O is a proprietary library provided by TELIT and in implementations is used in the development of BLUETOOTH communication between an ECG device 100 and the software application using a TERMINAL I/O profile.

CRYPTOSWIFT is an opensource library and may be used for standard and secure cryptographic algorithms implemented in SWIFT. MESSAGEPACK JAVA is a library which may be used to encode/decode messages communicated between an ANDROID version of the software application and an ECG device 100 over BLUETOOTH. MESSAGEPACK SWIFT is a library which may be used to encode/decode messages communicated between an iOS version of the software application and an ECG device 100 over BLUETOOTH.

The one or more servers 164 are in implementations responsible for accepting connection requests from the software application, receiving data from ECG devices 100 through the software application, and sending user commends to the ECG devices 100 through the software application. In implementations the software application relays the ECG device 100 setup parameters from the one or more servers 164 and relays notifications and ECG data from the ECG devices 100 to the one or more servers 164. The ECG device 100 records ECG data and establishes the BLUETOOTH connection with the user's computing device (using the software application) to send data to the one or more servers 164 and handle the commands from the one or more servers 164.

In implementations the communication between the ECG device 100 and the software application is done over BLUETOOTH Low Energy (BLUETOOTH LE) connectivity.

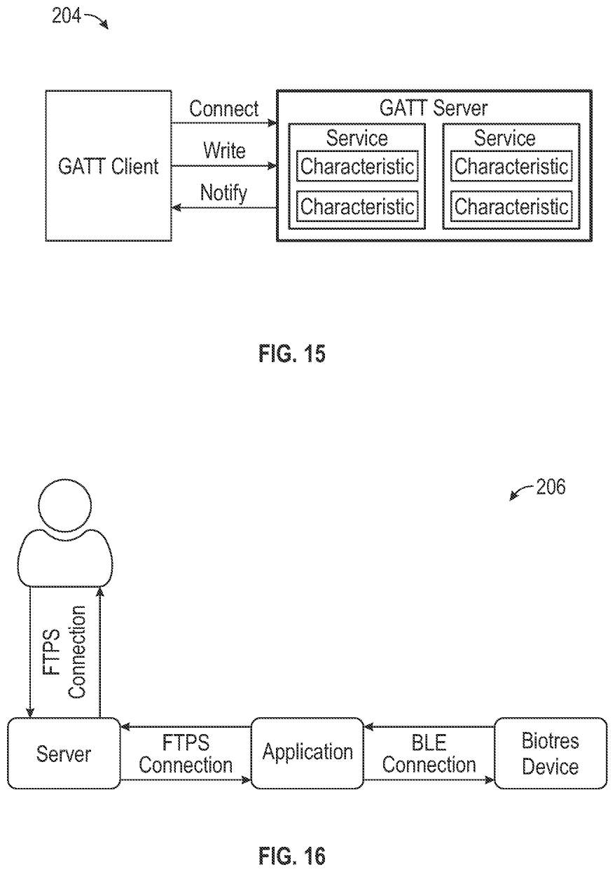

Referring to diagram 204 of FIG. 15, in implementations a BLUETOOTH LE connection is facilitated by and/or includes a BLUETOOTH Generic Attribute (GATT) server and a GATT client. The GATT server may store data values, which may be hierarchically structured in services and characteristics, while the GATT client may read and write these values and may be notified by the server with respect to value changes. Each characteristic may have its own properties and can have an optional Client Characteristic Configuration, which allows configuration of the characteristic (e.g., enable notifications). In implementations the BLUETOOTH modem or module of the ECG device 100 is provided by TELIT and a proprietary BLUETOOTH LE GATT profile developed by TELIT for bidirectional serial data communication called TERMINAL I/O is used as the communication profile between the software application and the ECG device 100.

Referring now to diagram 206 of FIG. 16, in implementations the communication between the software app and the one or more servers 164 is designed to be file-based communication in which commands which the one or more servers 164 want to send to the ECG device 100, and data and notifications which the ECG device 100 wants to send to the one or more servers 164, are exchanged as files. In implementations FTPS is used for the communication between the software app and the one or more servers 164. In implementations the software app is used to manage data communication between the ECG device 100 and the one or more servers 164. Under normal operation, when within the wireless range, the software app connects to the ECG device 100 over BLUETOOTH and establishes a secured data exchange with the one or more servers 164 using the computing device's WiFi or cellular or other wireless connection. The software app in implementations is required to relay the ECG device 100 setup parameters from the one or more servers 164, and to relay notifications and the ECG data from the ECG device 100 to the one or more servers 164. The ECG device 100 buffers or delays notifications and ECG data upload to the one or more servers 164 if the software app is not available due to any interruption in wireless communication. The user(s), through an FTP or FTPS client, can send commands to the ECG device 100 and download study data uploaded by the ECG device 100 through the software app.

In implementations communications between the ECG device 100 and the software app, and between the software app and the one or more servers 164, will be encrypted to protect PHI from exposure. AES may be used to encrypt the BLUETOOTH communications between the ECG device 100 and the software app, while Transport Layer Security 1.2 (TLS 1.2) may be used to encrypt the FTP or FTPS communications between the software app and the one or more servers 164.

In implementations a Model, View, View Model (MVVM) architecture is used for the software application. Model View Controller (MVC) and Model View Presenter (MVP) are the most commonly used design patterns within ANDROID and iOS software applications but there are tradeoffs/cons with these.

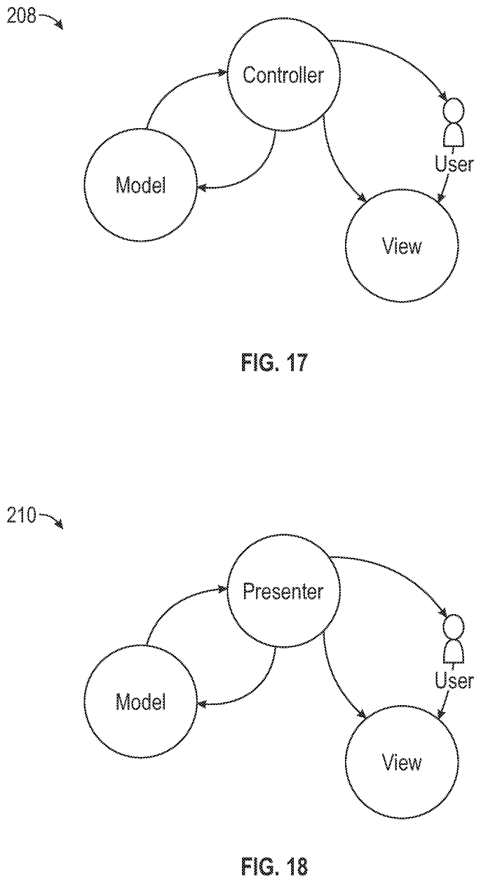

Referring now to diagram 208 of FIG. 17, an example MVC architecture is representatively illustrated. In this pattern/architecture the model represents the data models, manages the data states, and has business logistics. The view renders the user interface(s), sends the user input to the controller, and provides the layout/views. The controller receives/interprets/validates input, handles segues to other controllers, queries and updates models, creates and updates views, and handles activities/fragments. This model helps the developer easily/quickly understand to use and build/edit the application. All the objects in the application are assigned one of three roles: model, view, or controller. A problem arises when developers put the majority of their application logic in the view controller class. The view controller will do the heavy tasks and handle a lot of the work. As the application is expanded for increased/added functionality and more code gets transferred to the controllers, they become bloated and brittle. It is also hard to have the unit tested because all the logic code will be put in the view controller. Sometimes it is impossible to test without modifying the code.

Referring now to diagram 210 of FIG. 18, an example MVP architecture is representatively illustrated. In this pattern/architecture the model represents the data models, manages the data states, and has business logistics (the same as in the MVC pattern). The view will implement an interface for the presenter's actions and will handle activities/fragments/views/layouts. The presenter has no relation to the views (unlike MVC)— operations are invoked by our views and views are updated via a view's interface. In some ways the MVP pattern seems similar to the MVC pattern, but in some cases it is far better as the presenter performs the interface actions and has no knowledge of the views it is trying to update—it can therefore easily be tested. One disadvantage of using this model is in relation to maintenance. Just like controllers, presenters are prone to collecting additional business logic, sprinkled in over time. At some point, developers may find themselves with large unwieldy presenters that are difficult to break apart. Also, it may be a challenge when the application has to build a smooth interface, needs to update the views constantly/continuously, or when it is desirable to have no effect when the application configuration changes.

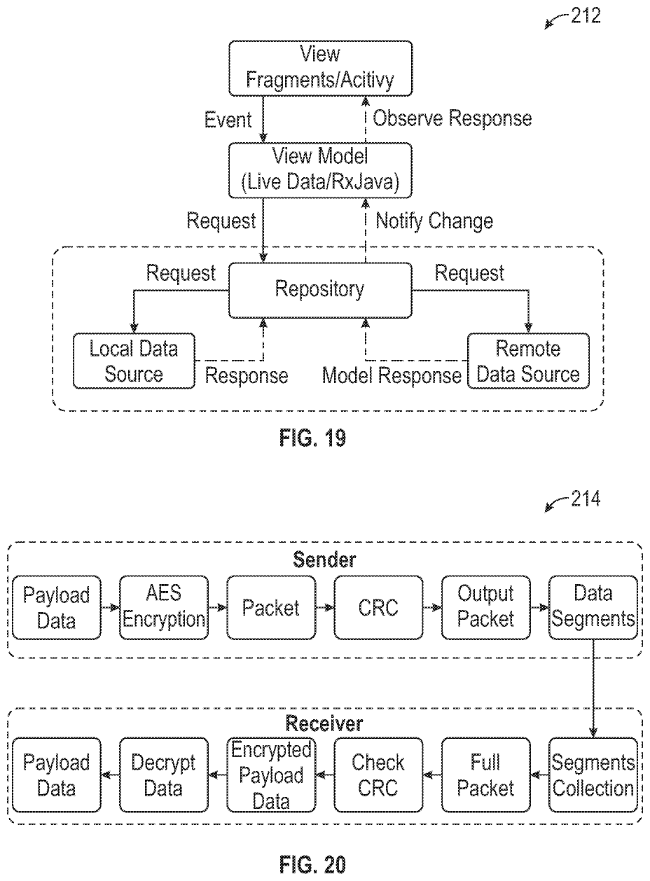

Referring now to diagram 212 of FIG. 19, an MVVM architecture/pattern is representatively illustrated. In this pattern the model handles/facilitates business logic, local and remote data source(s), and repositories. The view sends user actions to the view model and gets a response (view observes some data which view model exposes). The view model handles an interface logic center and has no reference to the view. Some advantages of the MVVM pattern are: there is no tight coupling between view and view model; no interfaces between view and model; easy to do unit testing and code is event-driven; smooth interface updating facilitated by data binding.

In implementations the software application uses an MVVM architecture/pattern to promote code separation, reusability, extensibility, testability, and a well-defined interface. There are still tradeoffs to this pattern. For example databinding is hard to debug when getting errors from layout files, and the pattern is hard to understand for the inexperienced developer.

Some architectural model elements will now be defined, including describing classes, their organization in packages and subsystems, and the organization of these packages and subsystems into layers. Some use-case realizations and related aspects of the architecture will also be described.

Using the MVVM architecture/pattern, all classes of the application may be divided into three main roles, as indicated below:

### 1. Model

a. Business logic: Device Handler class, Parsing Data class, AES Manager class, Device class, Device Manager class, BLUETOOTH client class, TCP client class, Packet builder, Command factory, Command class, Handlers class, Handler factory.
b. Local data source: User Entity class, Device Entity class, Room Database class.
c. Remote data source: User Interface class, User Model class, API Client class, Login Task class.
d. Service: Foreground Service class, Manager Service class.
e. Device Repository class, User Repository class, Core Repository class

### 2. View

a. Login screen, App information Screen, Dashboard Screen, Connect Screen, Base Screen.

### 3. ViewModel

a. Login ViewModel, Dashboard ViewModel, Connect ViewModel

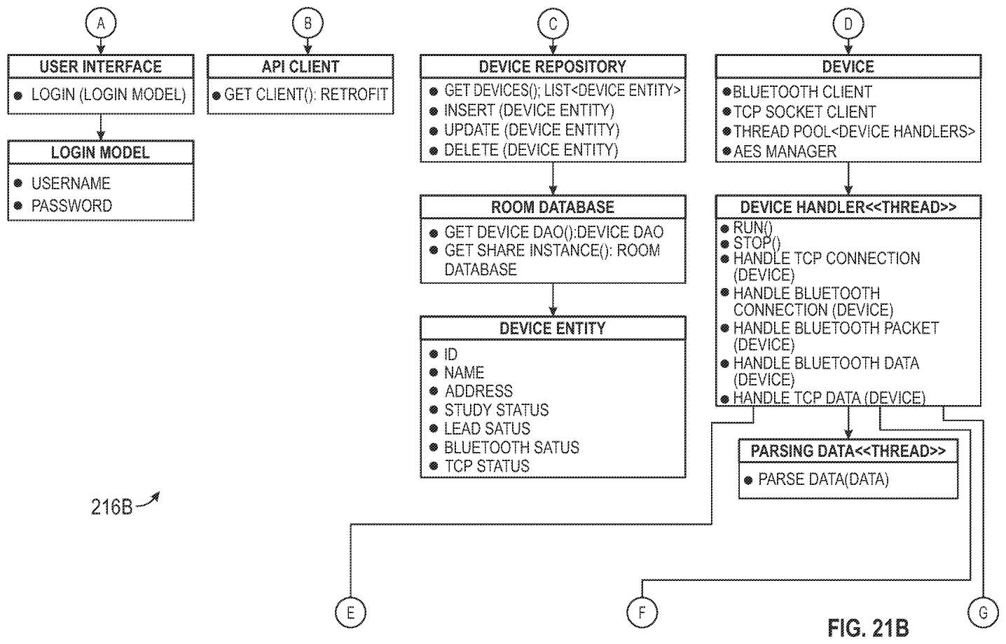

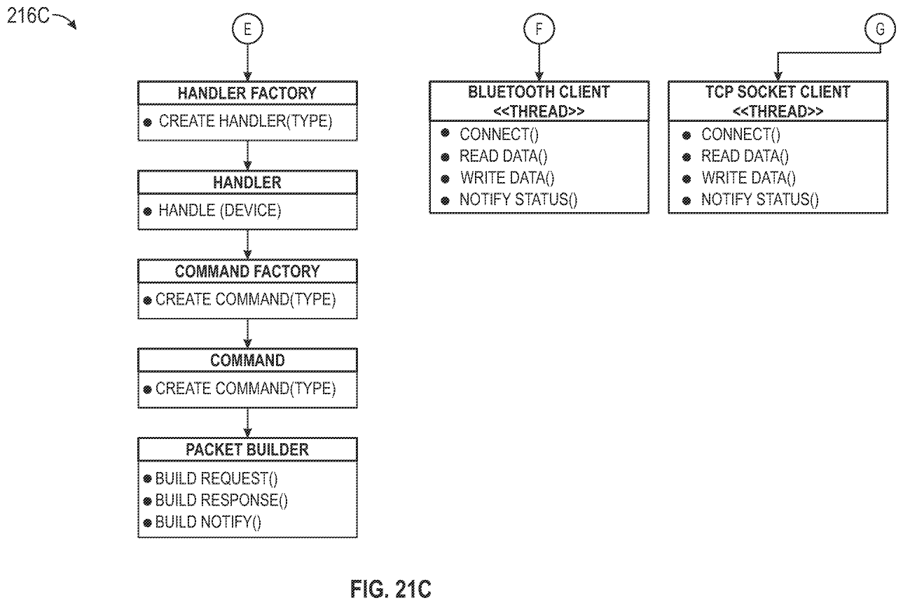

Diagrams 216A, 216B, 216C of FIGS. 21A, 21B, 21C, respectively, together representatively diagram and model example elements of the software application if implemented using an MVVM architecture/pattern.

The systems, methods, and devices disclosed herein may be configured to protect sensitive data (such as PHI and/or other healthcare data). This may include mechanisms to ensure that data stays uncorrupted and unchanged by persons without authority/right to make changes.

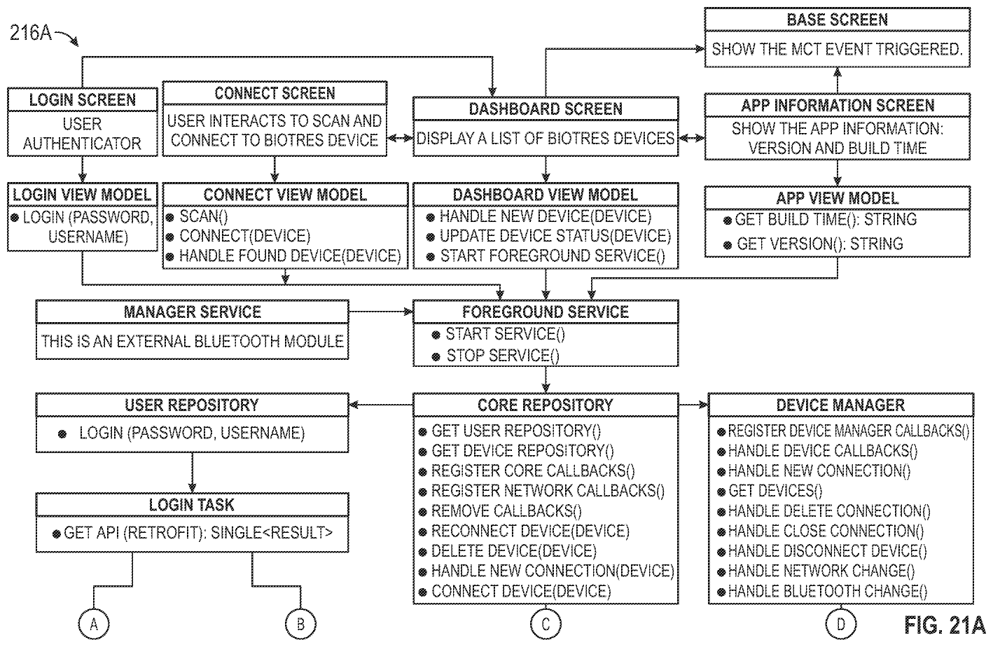

Referring now to diagram 214 of FIG. 20, before a sender sends data it may be encrypted by the AES algorithm and then be added into the packet. The whole packet may be calculated by a 17-bit cyclic redundancy check (CRC-16). The result of the CRC calculation may be put into the packet to maintain the consistency, accuracy, and trustworthiness of data over its entire communication life cycle. If the packet is big, it will be divided into many segments, serialized, and sent across the network. The receiver side will collect all of the segments to capture the full packet and use the serialization to organize them in proper order. All packets sent over the network may be checked with a CRC-16. If no error is detected, the payload data may be taken from the packet and decrypted by the AES algorithm. The original payload data will be received from the output of the decryption processing.

In implementations ECG device 100 will not store any personal or sensitive data of the patient and will have no stored memory of the data or the information transferred through it. In implementations the ECG device 100 and the one or more servers 164 will have a protocol to communicate with one another.

For the one or more servers 164 and/or the software application to handle multiple ECG devices 100 sending and retrieving data constantly and continuously, the one or more servers 164 and/or software application may be configured to handle multiple commands at the same time. This may involve using multiple threads for handling the long running processes and concurrent tasks. Using multiple threads may increase speed by allowing the one or more servers 164 and/or software application to handle multiple things at the same time. Multiple threads help ECG system 162 achieve parallelism. Modern computer processing units (CPUs) are very fast and often contain multiple cores. Using a single thread fails to take advantage of all the cores, resulting in idle hardware. By using threads, the one or more servers 164 and/or software application can take full advantage of multiple cores to improve response time. The one or more servers 164 and/or software application may use many threads managed by a thread pool service for parsing data, handling data, sending data, and retrieving data. The one or more servers 164 and/or software application may use RXJAVA/RXSWIFT to create, manage, and operate many tasks.

The binary serialization format MESSAGEPACK may be used for packet encapsulation. This allows data exchange among multiple languages such as JSON but is smaller (using less bytes) than a normal JSON formatter and is faster to parse or encode.

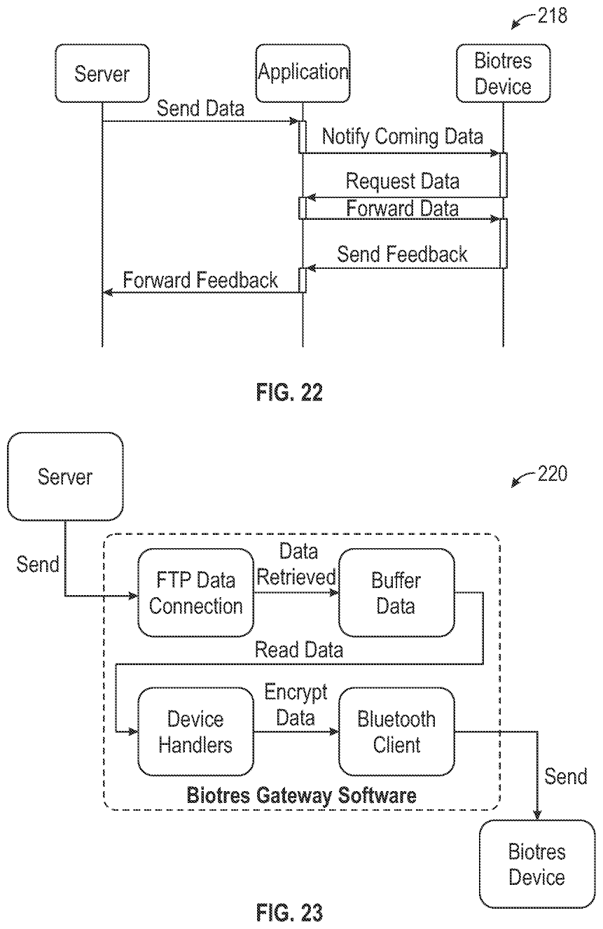

Diagram 218 of FIG. 22 representatively illustrates a process of sending/forwarding data from one or more servers 164 to the ECG device 100 through the software application, and diagram 220 representatively illustrates a process of handling data sent from the server. As the server wants to send data to the ECG device 100 through the software application over the FTP or FTPS connection, the software application will receive the data from the FTP or FTPS connection and then start the process of handling data. In this role, the software application is working as a gateway to forward data and has no knowledge of the information sent from the server to the ECG device 100. After processing the data, the software application will push a notification over BLUETOOTH to the ECG device 100 to announce the coming data. The ECG device 100 will request to read a slice/set/portion of the data when it is available. The software app will forward a slice/set/portion of data to the ECG device 100.

One reason for buffering data from the one or more servers 164 and sending a slice/set/portion of the data frame to the ECG device 100 is that the server may send large amounts of data that may be greater in size than the memory capacity (or remaining memory capacity) of the ECG device 100. Moreover, the ECG device 100 acquires ECG data continuously, and this data capture is a high priority. Further, at times the ECG device 100 will be busy capturing ECG data or performing other functions when the server data comes, and so will not be immediately available to receive it.

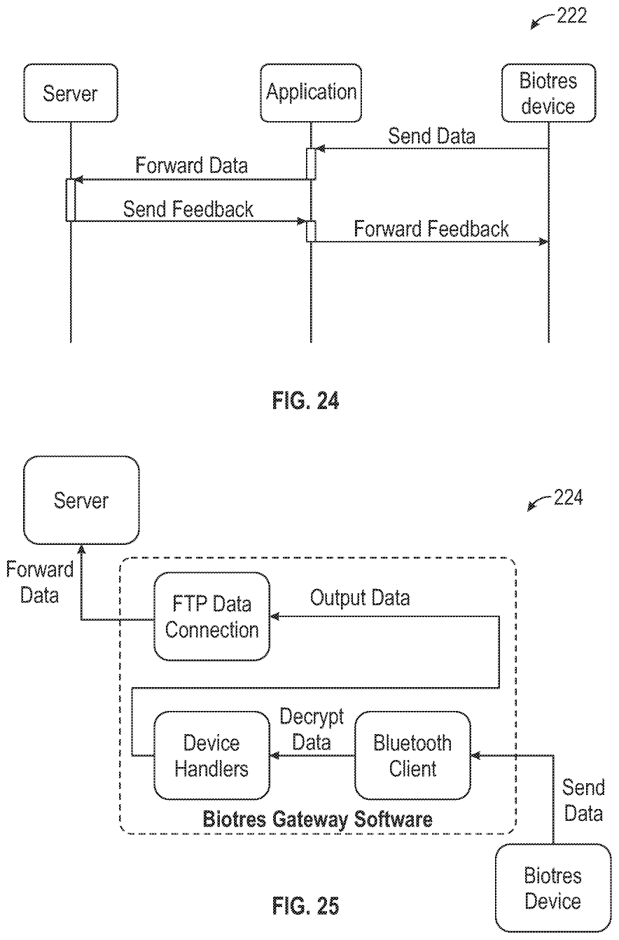

Diagram 222 of FIG. 24 representatively illustrates a process of sending data from the ECG device 100 to the one or more servers 164, and diagram 224 of FIG. 25 representatively illustrates a process of handling data from the ECG device 100. As an ECG device 100 wants to send data to the one or more servers 164, the software application will receive the data from the BLUETOOTH client over the BLUETOOTH connection. The retrieved data will be sent directly to the one or more servers 164 over an FTP or FTPS connection. In implementations the software application will not know the contents of the forwarded information. In implementations the data could be encrypted both when sent from the ECG device 100 to the software application and when forwarded by the software application to the one or more servers 164 (such as after decrypting and then re-encrypting the data).

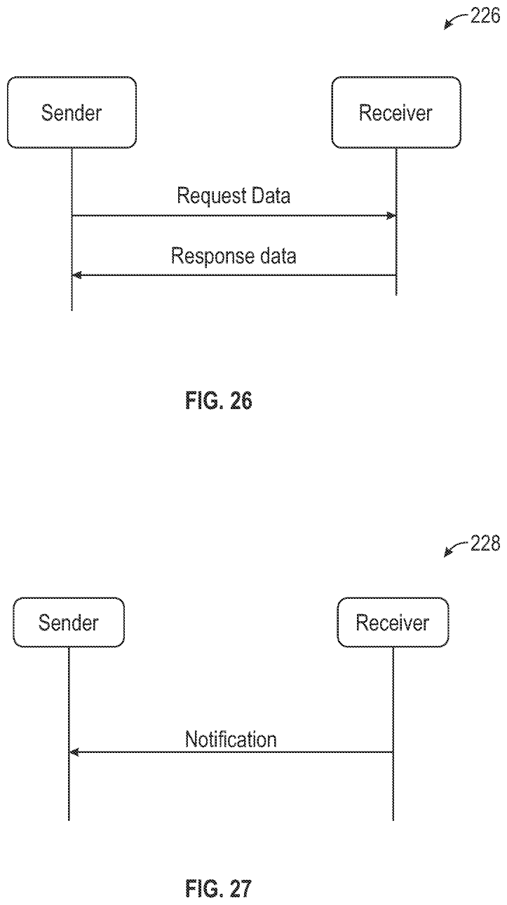

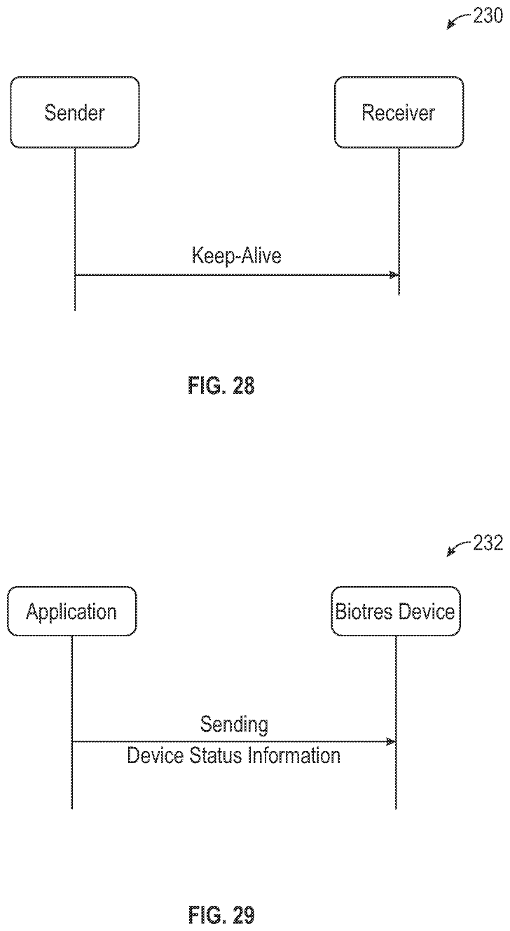

Diagrams 226, 228 and 230 of FIGS. 26, 27, and 28, respectively, representatively illustrate three example communication flows between the software application and the ECG device 100. A request/response flow (diagram 226), a notification flow (of diagram 228, and a keep-alive flow (of diagram 230). In the request/response flow the requester sends a request packet and waits for a corresponding packet from the responder. This flow is used to make sure a request packet is received and processed successfully by the responder. The requester will receive a response packet with result(s) and additional information.

In the notification flow the sender sends status or data to the receiver without response. The sender does not care/know if the packet reaches the responder or not. This flow is suitable for streaming or uploading a large amount of data because it can make use of the highest bandwidth.

The keep-alive flow is used (for example an occasional packet sent) to maintain the connection between the application and the ECG device 100. The sender does not care/know if the packet reaches the receiver or not.

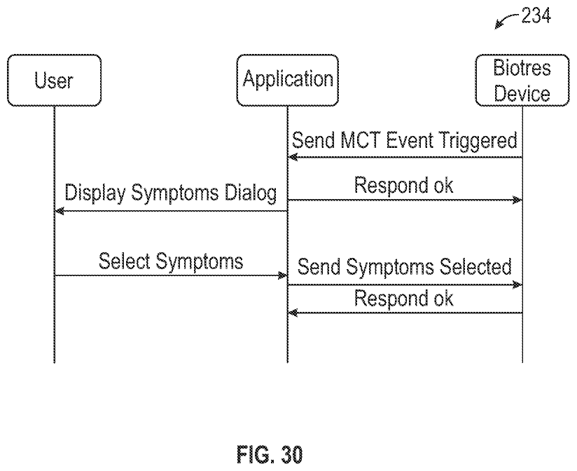

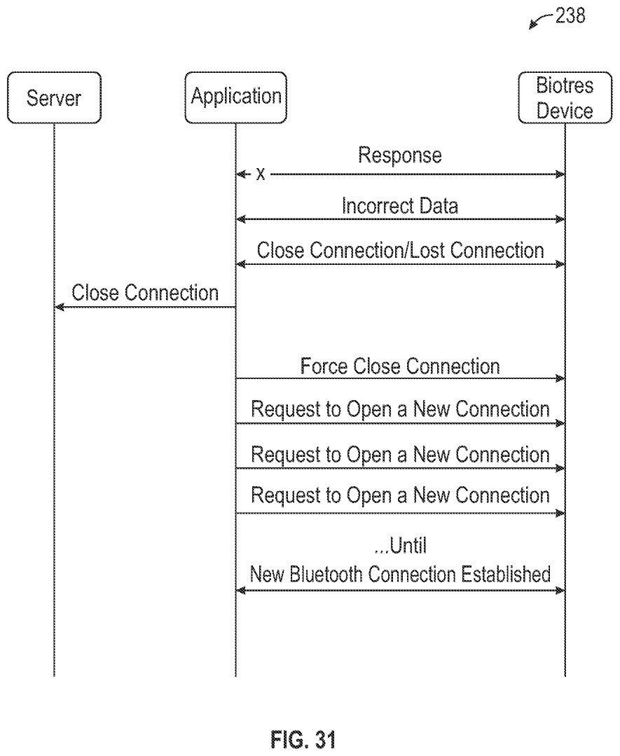

In implementations the software application has the responsibilities of displaying the status of the ECG device 100 and showing the mobile cardiac telemetry (MCT) event. Diagram 232 of FIG. 29 representatively illustrates device status flow and diagram 234 of FIG. 30 representatively illustrates MCT event flow. In implementations the device status includes battery status, electrode status, and study status, which are displayed to the user using one or more user interfaces of the software application. The ECG device 100 may use the notification flow to update the status every minute or immediately if one thing has changed. As the user wants to select the symptoms for a manual MCT event triggered from the ECG device 100, pushing the marking switch 104 will send a signal to the software application. The software application will then display, on one or more user interfaces displayed on the user's computing device, a symptoms event dialog (such as user interface 154) which in implementations may be terminated after a predetermined amount of time (such as 20 seconds). The user selects the symptoms and this data is sent back to the ECG device 100. The selected symptoms will be sent to the one or more servers 164 for storing and analyzing.

Diagram 238 of FIG. 31 representatively illustrates elements of maintaining and managing an ECG device 100 connection. In implementations the software application is working as the active side when maintaining the BLUETOOTH connection with the ECG device 100. The software application may force close a current connection if a response cannot reach it or if the receiving/response data is invalid. When the connection is lost the software application may attempt to reconnect with the ECG device 100 every thirty seconds (or on some other predetermined time interval) until the connection is established again. The software application also may disconnect with the one or more servers 164 when the ECG device 100 connection is disconnected.

Although it has been disclosed that the communication between ECG device 100 and the software application may utilize BLUETOOTH Low Energy connectivity (through a user computing device), in implementations this will change as the ECG device will have its own cellular connectivity or other connectivity inherent to the ECG device itself such that it may communicate with remote servers without going through a computing device. Additionally, although it is disclosed that the ECG device buffers or delays notifications and ECG data upload to remote servers if the software app is not available due to any interruption in wireless communications (such as between the ECG device and the user's computing device or between the user's computing device and remote servers), in implementations in which the ECG device itself has its own cellular or other mobile connectivity to remote servers such buffering may not be needed or may be obviated. Additionally, although FIGS. 26-28 are described as illustrating communication between the ECG device 100 and the software application, in implementations wherein ECG devices have their own integral cellular or other connectivity these drawings may instead representatively illustrate communications between the ECG device 100 and the one or more servers 164.

As described above, an electrocardiogram (ECG) is a graphical record of electric potentials generated by the heart muscle during each cardiac cycle. These potentials may be detected on the surface of the body using electrodes attached to the chest. In implementations ECG device 100 amplifies and records these signals/potentials continuously for up to thirty days (or in some cases more or fewer days). The ECG recording data is sent securely to one or more servers 164 (e.g., via FTPS) through the software application in MIT16 data format. Though secure file transfer and/or FTPS is one option, this can change in a major way.

As indicated above, an optional BIOTRES Gateway Application (software app) is available to the patient during ECG recording to provide status indications relative to the ECG device 100 (e.g., battery level, electrode connection on/off, study state such as running/paused/completed, and so forth) by displaying this information on one or more user interfaces on the patient's computing device. The software app allows patients to identify their symptoms (such as palpitations, shortness of breath, dizziness, chest pain, pressure, or other) when/after pressing the marking switch 104. In implementations, however, no ECG data is displayed to the patient. The software application allows/facilitates uploading of the device's status indications, the patient's selected symptoms, and ECG data, any of which may be uploaded in partial segments intermittently or occasionally during the study to speed the final upload process at the end of the patient study. The software application is not required for the patient but may be useful to the patient for viewing the device status indications and/or other information, and for indicating symptoms during an event, on his/her computing device.

As also indicated above, prior to initiating ECG recording a clinician may use the software app on a BLUETOOTH (or other wireless communication protocol) enabled computing device to set up the ECG device 100 at the beginning of a study (this is done in conjunction with the one or more servers 164 at least intermittently communicatively connected with the clinician's computing device inasmuch as data related to the study, the patient, the clinic, etc. are stored and associated with one another in the one or more servers 164 during setup). During setup the clinician uses privileged login credentials to log into the software application to ensure that the ECG device 100 is communicatively coupled with the software application on the clinician's computing device and to ensure that the ECG device is communicatively coupled with the one or more servers 164 via the software application on the clinician's computing device. In some implementations server connection status indicator 140 could instead indicate that the respective computing device is communicatively coupled with the one or more servers 164, and not necessarily the ECG device 100, in any given moment. Server connection status indicator 140 may also indicate, in some implementations, that a communicative connection was successful the last time attempted (or within a certain time frame, e.g., within the last ten minutes) as opposed to indicating in real-time that a communicative coupling is currently live/active. The clinician uses the privileged login credentials to connect his software app and/or the ECG device 100 with the one or more servers 164 and uploads a file to initiate a study on the ECG device 100. The study then initiates using the ECG device 100 and the software application using the information provided to the ECG device 100 by the one or more servers 164.

In implementations, the clinician may also train the patient on the use of the ECG device 100 and place the device on the patient prior to starting the study through the software application. After the study is complete the patient may return the ECG device 100 do the clinician, who will clean it, ensure that it is connected to the software application on the clinician's computing device, and ensure a connection of the ECG device 100 with the server is established (via the clinician's computing device/software application) so that the ECG recorded data may be uploaded. Once the recorded ECG data is uploaded it will stored in MIT16 format (or in any other useful format) using the one or more servers 164 and will be accessible/downloadable by authorized parties, such as the clinician.

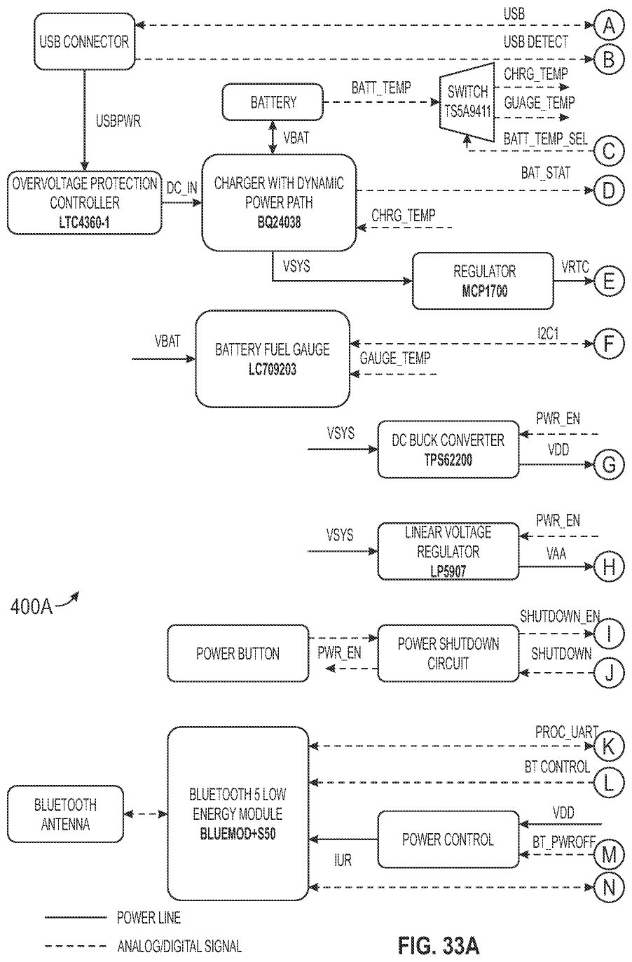

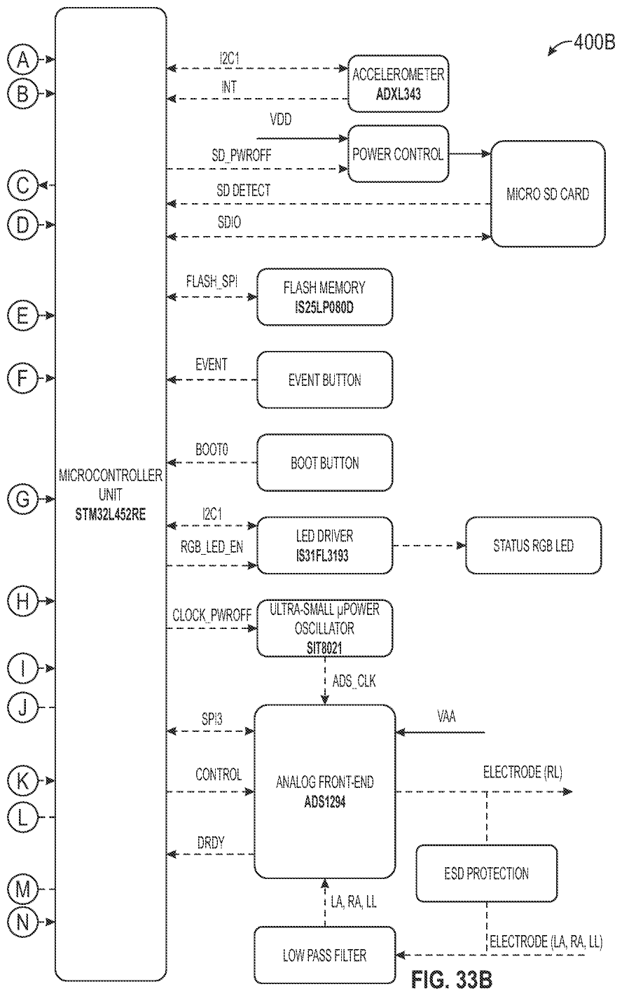

FIGS. 33A-33B representatively illustrate example hardware architecture, and related power lines and signals, for ECG device 100. This is not necessarily a comprehensive view of all hardware architecture of ECG device 100, but the practitioner of ordinary skill in the art will know how to build, configure, and use the ECG device 100 using the details provided. The diagrams include example model numbers for some hardware elements, which model numbers may be used to determine the manufacturer/provider of the respective element.

Some benefits of the disclosed ECG systems and devices and related methods are further detailed in TABLE 1 below.

TABLE 1
Benefits of ECG Systems, Devices and Related Methods

| | | | Compared to |
|---|---|---|---|
| | Value to Healthcare | | Existing |
| Feature | Provider/Clinician | Value to Patient | Technologies |
| Small and | Small size and ease of use | Patients wear the | Superior |
| discreet | leads to better patient | small, lightweight | |
| | compliance | ECG device | |
| | | comfortably, without | |
| | | added bulk | |
| Marking switch | Never again wonder what | No manual diary to | Superior |
| with app-based | patients are experiencing | pull out when | |
| diary available | when they push the marking | patients feel | |
| through | button-they can choose | symptoms-just a | |
| patient's smart | symptoms on the software | touch of their phone | |
| phone or other | app | screen (or other | |
| computing | | computing device) | |
| device | | logs their symptoms | |
| 3 channels | Built-in redundancy ensures | Patients get their | Unique |
| | an accurate study | studies done only | |
| | | once-repeats are rare | |
| Reusable | Cardiologists supply the ECG | A patients can get | Unique |
| | device from their own | the ECG device right | |
| | inventory of devices, the | at the cardiologist's | |
| | reusable devices are returned | office during the | |
| | to the practice upon | initial appointment | |
| | completion of the study for | | |
| | redeployment. | | |
| Supports | Placement/removal of ECG | EOBs no longer | |
| practice growth | device, data analysis, review | reference unknown | |
| | and interpretation are not | providers | |
| | outsourced to a third party, | | |
| | but stay within the practice, | | |
| | no more calls from patients | | |
| | wondering why their | | |
| | explanation of benefits | | |
| | (EOBs) reference an unknown | | |
| | third party provider | | |
| Faster | Allows cardiologists to more | Reduces patient wait | Unique |
| turnaround | efficiently manage their | and associated | |
| time for results, | patients' condition(s) | anxiety | |
| reduces time to | | | |
| review results | | | |
| from weeks to a | | | |
| few days | | | |
| Standard | Ease of use-no need for | Patient can change | Unique |
| electrodes | unique supplies | electrodes during | |
| | | study and doesn't | |
| | | have to make a | |
| | | patch/electrode last | |
| | | 14 days | |
| Storage | Recording up to 1 year, in | | |
| | implementations | | |

As indicated herein, the ECG devices 100 provide 3-channel collection and recording. In implementations they are wearable Holter patch devices that can provide continuous 3-channel recording of ECG (heart) data. All other known Holter patch devices are 1 channel or 2 channels. Due to the rechargeable battery, the ECG device 100 can be worn continuously for forty-eight hours, in connectivity mode, before needing to be charged for one hour, enabling continuous data collection for extended periods of time without any intervention, a distinction not possible with traditional Holter patch solutions. Because the disclosed systems and devices utilize wireless connectivity, such as BLUETOOTH, to offload data, time to diagnosis may be reduced. Current Holter patch solutions can take up to a week before diagnoses are available due to manual data downloading and a lack of connectivity. The systems, devices and methods (and software apps) disclosed herein are further easy to understand and navigate. The ECG devices 100 are comfortable to wear during regular day-to-day activities. The ECG devices 100 and related systems, including the software app, further utilize a modular design such that there is flexibility to support adding features and functionality to the ECG devices 100, ECG systems 162, software app, and other disclosed elements.

In implementations other elements may be added to the disclosed ECG devices, systems and methods, such as hardware and/or software elements for detecting/determining respiration, temperature, motion/activity (such as accelerometer and/or gyroscopic sensors), and any derivative biometrics (for example heart rate may be a derivative of electrocardiogram sensing/measurements, fall detection and sleep detection may be derivatives of motion/activity measurements, etc.). Associated user interfaces may display such measured or derived biometrics.

Upon information and belief the ECG device 100 is the only existing 3-channel 3-contact-point ECG device. There are existing ECG devices that require four or five contact points to create/collect three channels.

The various devices and/or assemblies disclosed herein and their elements, sub-elements, sub-assemblies, and so forth may be formed from any materials that will feasibly allow, facilitate, and/or otherwise not hinder their respective functions as described herein. For example, any of the devices, elements, or sub-elements may, wherever possible, be formed of metals, polymers, composites, ceramic materials, fabrics, and so forth. In implementations the housing 102 is a patient-contacting element (specifically second housing portion 102B) and may be formed of a polycarbonate or acrylonitrile butadiene styrene (ABS) such as BAYBLEND M850 XF. The leads 112 may be patient-contacting elements inasmuch as portions of them may occasionally contact the user during movement notwithstanding there being an electrode between the leads and the user's skin. In implementations the portions of the leads that may contact the user may be formed of thermoplastic polyurethane (TPU), polyvinyl chloride (PVC), acrylonitrile butadiene styrene (ABS), or polyamide 66 (PA66).

Furthermore, there are a variety of ways in which the various elements may be directly or indirectly coupled together. Notwithstanding the specific ways in which elements are depicted as being coupled together herein, these same elements could, wherever feasible, be joined together in any of the following ways: manually removably coupled together such as using a friction fit, hook and loop fasteners, a reusable adhesive, manually removable bolts and nuts or screws or other threaded fasteners, and any other type of manually removable coupling mechanism; or fixedly/permanently coupled together such as using a permanent adhesive, rivets, welding, melt joining or heat bonding, and any other type of permanent coupling mechanism that is not manually removable. Manually removable, as defined herein, refers to the ability to remove a coupling using manual force either using hands alone or using non-powered hand tools.

The above-described elements may in implementations be configured or arranged in a variety of arrangements, each arrangement with its own advantages as will be understood by the practitioner of ordinary skill in the art, notwithstanding the specific example arrangements which are discussed above and representatively illustrated in the drawings.

Furthermore, while each individual above-described element may be configured as shown in the drawings and/or as discussed above, these are only representative examples and other configurations are possible for any individual element, with various advantages and tradeoffs as will be understood by the practitioner of ordinary skill in the art.

In places where the phrase “one of A and B” is used herein, including in the claims, wherein A and B are elements, the phrase shall have the meaning “A and/or B.” This shall be extrapolated to as many elements as are recited in this manner, for example the phrase “one of A, B, and C” shall mean “A, B, and/or C,” and so forth. To further clarify, the phrase “one of A, B, and C” would include implementations having: A only; B only; C only; A and B but not C; A and C but not B; B and C but not A; and A and B and C.

In places where the description above refers to specific implementations of electrocardiogram systems and devices and related methods, one or more or many modifications may be made without departing from the spirit and scope thereof. Details of any specific implementation/embodiment described herein may, wherever possible, be applied to any other specific implementation/embodiment described herein. The appended claims are to encompass within their scope all such changes and modifications as are within the true spirit and scope of this disclosure.

Furthermore, in the claims, if a specific number of an element is intended, such will be explicitly recited, and in the absence of such explicit recitation no such limitation exists. For example, the claims may include phrases such as “at least one” and “one or more” to introduce claim elements. The use of such phrases should not be construed to imply that the introduction of any other claim element by the indefinite article “a” or “an” limits that claim to only one such element, and the same holds true for the use in the claims of definite articles.

Additionally, in places where a claim below uses the term “first” as applied to an element, this does not imply that the claim requires a second (or more) of that element—if the claim does not explicitly recite a “second” of that element, the claim does not require a “second” of that element. Furthermore, in some cases a claim may recite a “second” or “third” or “fourth” (or so on) of an element, and this does not necessarily imply that the claim requires a first (or so on) of that element—if the claim does not explicitly recite a “first” (or so on) of that element (or an element with the same name, such as “a widget” and “a second widget”), then the claim does not require a “first” (or so on) of that element.

Method steps disclosed anywhere herein, including in the claims, may be performed in any feasible/possible order. Recitation of method steps in any given order in the claims or elsewhere does not imply that the steps must be performed in that order—such claims and descriptions are intended to cover the steps performed in any order except any orders which are technically impossible or not feasible. However, in some implementations method steps may be performed in the order(s) in which the steps are presented herein, including any order(s) presented in the claims.

The methods, systems, and devices disclosed herein can be implemented using a variety of computing environments and devices, including personal computers, server computers, mobile devices, smart speakers, data stores, database servers, databases, web servers, smart watches, smart glasses, tablets, laptops, a telecommunications network (such as the Internet), etc.

ECG system 162 may include, in implementations, any general-purpose computing devices and specific custom computing devices suitable for use in performing or facilitating the functions described herein. Any of the computing devices may include one or more: processors, memories (e.g., random-access memory (RAM) and/or read-only memory (ROM)), software elements, and any additional components which may be implemented as hardware and/or software (such as, by non-limiting example, a receiver and transmitter for communicating with other computing devices of the system locally and/or through a telecommunications network, wireless communication elements, one or more microphones and/or speakers for audio input/output, one or more displays for presenting information in visual format and/or for user interfaces, a text-to-speech synthesizer for converting text to speech, a speech-to-text converter, user input elements such as a keyboard, keypad, touchscreen, mouse, and so forth). The double-arrowed connectors of FIG. 12 representatively illustrate the individual elements being able to communicate with one another directly (and/or indirectly through other elements) wirelessly and/or through wired communicative couplings.

It should be noted that portions of the systems, devices, and methods and methods disclosed herein may be implemented using software and/or a combination of software and hardware, e.g. using application specific integrated circuits (ASIC), general purpose computers, or any other hardware equivalents. In one embodiment, one or more or all of the discussed methods may be implemented using software loaded into memory and executed by a processor to implement the functions discussed herein. As such, in implementations one or more or all of the methods disclosed herein may be implemented using one or more non-transitory computer-readable storage media, e.g., one or more RAM memories, one or more magnetic or optical drives or diskettes, and/or so forth.

Further, ECG system 162 may have additional elements not explicitly shown. For example, another computing device may be at least temporarily communicatively coupled with the one or more servers 164 and may be used by an administrator (e.g., not a clinician or patient) to populate a data store (such as a database) with some of the elements of the system, and to make changes to the database elements, and/or to edit elements/features/design of the software application provided by the one or more servers 164, and so forth. In implementations data stores other than databases may be used. As used herein, a database is considered a subset of a data store. The administrator's computing device could be coupled with the one or more servers 164 directly, though also or alternatively through network 170 which may be, by non-limiting example, the Internet, though it could also be a local area network (LAN) in a more localized deployment of the system. The admin device could also communicatively couple with the one or more servers 164 through a web server, such as by accessing a website using credentials, or by an admin version of the software app made accessible using an application server of the one or more servers 164. The administrator computing device could be a desktop computer, laptop, mobile phone, tablet, smart watch, smart speaker, smart glasses, and/or any other computing device.

In implementations an admin computing device, database and/or database server, web server, app server, and the like could all be implemented on a single machine (such as using virtual servers on a single machine) but in large network deployments there will more likely be a plurality of cloud-connected server racks used to implement these various elements as the number of users scales up. For example, AMAZON WEB SERVICES (AWS) server racks may be utilized to implement one or more database servers, databases, app servers, web servers (if desired), and so forth so that the number of users may be increased to very large numbers.

The administrator, clinicians, patients and/or other users could access elements of the system through one or more software applications on a computer, smart phone, tablet, or any other computing device, such as through one or more software applications as described above (such as by non-limiting example a mobile app having interactive user interfaces) facilitated at least in part by one or more application servers.

In implementations the system 162 may interact with third party server(s)/database(s) to gather and/or store/retrieve information and/or to at least partially perform one or more of the functions disclosed herein. This may allow the methods and/or functions as disclosed herein to be at least partially delegated and/or performed by third parties using their own servers and/or hardware/software.

System 162 may have a number of user computing devices simultaneously communicatively coupled with the one or more servers 164 via the telecommunications network, including one or more: personal computers; mobile phones; smart glasses; mobile tablets; smart watches; smart speakers; robots; and any other human interaction device which may include microphone/speaker and/or visual input/output and/or other input/output elements for user interaction (by non-limiting example, user interfaces integrated in land/sea/air vehicles, phones, interactive displays, wearable technology, and any other human interaction device now known or hereafter discovered/invented).

The system 162 at any given moment may have fewer or more user computing devices communicatively coupled with it, and each type of device may be scaled up to any number of users—FIG. 12 is accordingly a simplified diagram. For example, in implementations there may be thousands of users, or more, simultaneously interacting with the system through computing devices 166 and/or 168. The system and methods disclosed herein may interact with third party software/elements on the user computing devices (which third party software elements may themselves access remote servers and databases and the like). For example, GOOGLE ASSISTANT, AMAZON's ALEXA, or APPLE's SIRI may interact with the system and/or software app to help facilitate the functions disclosed herein. By non-limiting example, a patient having difficulty using touchscreens, such as because of slow or low mobility, may instead vocalize which symptoms he/she is experiencing, and the software app or native functions of the patient's computing device or a smart assistant or other app on the computing device may function to fill in or select the symptoms expressed vocally by the patient. This may be done in ways that the patient's PHI is never known or transmitted to any third party apps or to the computing device, so as to protect the patients PHI. System 162 may also have other elements for implementing the methods, in addition to those that are shown in the drawings and described herein.

While various embodiments of the systems and methods have been described above at a high level, these have been presented by way of example only, and not by way of limitation. Thus, the breadth and scope of a preferred embodiment of any system or method disclosed herein should not be limited by any of the above-described exemplary embodiments.

As used herein, the term “of” may refer to “coupled with.” For example, in some cases a display may be referred to as a display “of” a first computer or computing device, a display “of” a second computer or computing device, and so forth. These terms are meant to be interpreted broadly so that a display “of” a computing device may be a separate display that is, either by wired or a wireless connection, communicatively coupled with the computing device.

The phrase “computing device” as used herein is meant to include any type of device having one or more processors and capable of communicating information using one or more integrated or communicatively-coupled displays, such as a personal computer, a laptop, a tablet, a mobile phone, a smart phone, a personal data assistant (PDA), smart glasses, a tablet, a smart watch, a smart speaker, a robot, any other human interaction device, and so forth.

It is pointed out that the provider of a software application, to be installed on end user computing devices (such as, by non-limiting example, mobile devices) at least partially facilitates an at least intermittent communicative coupling between one or more servers (which host or otherwise facilitate features of the software application) and the end user computing devices. This is so even if the one or more servers are owned and/or operated by a party other than the provider of the software application.

In implementations the marking switch is at least partially translucent, the status indicator light is housed at least partially within the housing, and the status indicator light provides visual indications of ECG device statuses by shining one or more colors and/or flashing light patterns through the marking switch.

Although the user interfaces described herein are presented as being accessible through a software application installed on a computing device, in implementations they may alternatively or additionally be accessible using a browser (for example provided on one or more user-accessible website portals, which may be password-protected and accessed using a browser on a computing device).

In implementations the leads are rigid and extend away from the housing. As used herein, the term “rigid” is defined herein as having an elastic modulus at least 500 MPa or greater at room temperature. In other implementations “rigid” may be defined herein as having an elastic modulus as least as great as each of TPU, PVC, ABS, and PA66 at room temperature. In other implementations “rigid” may be defined as having an elastic modulus at least 100 MPa or greater at room temperature. In other implementations “rigid” may be defined herein as having no bending under manual force of a user that is noticeably visible to the naked eye.

In implementations the marking switch and power switch are formed of rubber.

Although specific workflows are disclosed herein (such as for setting up an ECG device, beginning a study, communicatively connecting the ECG device with one or more servers and with one or more computing devices, and so forth, these are only representative examples and can change from implementation to implementation.

In implementations the ECG device 100 and/or the one or more servers 164 may include one or more neural network chips for signal processing. The ECG device 100 and/or ECG system 162 may use machine learning (ML) and artificial intelligence (AI) on the server side using the one or more servers 164 (and/or locally on the ECG device 100) for data processing and/or to provide its own diagnoses. In implementations the methods disclosed herein may include, using the one or more servers 164 and/or one or more processors of ECG device 100, training an ML model to process and analyze collected heart-related data and to provide one or more diagnoses, possible diagnoses, conclusions, or possible conclusions based on the heart-related data.

In implementations software and/or hardware included in the ECG device 100 and/or the one or more servers 164 includes an ECG classifier. The ECG classifier may use algorithms, neural network algorithms, machine learning (ML), and/or artificial intelligence (AI) to analyze ECG data, such as in MIT16 or any other ECG format with one or more ECG channels. The ECG classifier may also be called an ECG classifier engine/module, ML engine/module/model, or AI engine/module/model. In implementations the ECG classifier is configured to determine the placement in time of the R peak of a heart beat and its type of beat (Normal, Ventricular, Supraventricular, and questionable). The ECG classifier may also determine arrythmias such as bradycardia, tachycardia, atrial fibrillation, atrial flutter, pause/asystole, ventricular couplet/runs/bigeminy/trigeminy/quadgeminy, supraventricular couplet/runs/bigeminy/trigeminy/quadgeminy, and heart blocks. The ECG classifier may also provide commentary/diagnosis explaining what the state of the provided data contains. The ECG classifier also output analysis containing heart rate variability (HRV) statistics, ST segment statistics, and QTC statistics.

P, Q, R, S, and T define segments or waves of a heartbeat. Accordingly, as used herein the terms QTC and ST are not acronyms. Rather, the QT interval refers to the time from the start of the Q wave of a heartbeat to the end of the T wave of the heartbeat (while QTC represents QT corrected for heart rate). ST similarly is not an acronym, rather the ST segment connects the QRS complex and the T wave of the heartbeat and accordingly refers to the time between the QRS complex and the T wave. The R peak is similarly a peak of the R wave of a heartbeat.

What is claimed is:

1. An electrocardiogram (ECG) device, comprising:
a housing;
a plurality of leads coupled with the housing and including couplers configured to attach to electrodes; and
a marking switch coupled with the housing;
wherein the ECG device is configured to monitor cardiac-related activity and record related data; and
wherein the marking switch is configured to, when engaged by a user, record a heart-related event.

2. The ECG device of claim 1, wherein the ECG device comprises no more than three leads.

3. The ECG device of claim 1, wherein the ECG device monitors the cardiac-related activity via no fewer than three channels.

4. The ECG device of claim 1, wherein the couplers comprise snap connectors.

5. The ECG device of claim 1, wherein the leads are rigid and extend away from the housing.

6. The ECG device of claim 1, further comprising a status indicator light configured to visually indicate a plurality of statuses of the ECG device.

7. The ECG device of claim 6, wherein the status indicator light is housed at least partially within the housing, wherein the marking switch is at least partially translucent, and wherein the status indicator light visually indicates the plurality of statuses by shining through the marking switch.

8. The ECG device of claim 1, wherein the electrodes comprise one of: rectangular electrodes having largest dimensions of no more than four inches by four inches; and circular electrodes having diameters of no more than four inches.

9. The ECG device of claim 1, wherein the ECG device is configured to be secured to the user using only adhesion of the electrodes to skin of the user.

10. An electrocardiogram (ECG) system, comprising:
an ECG device configured to monitor cardiac-related activity and record related data, the ECG device at least intermittently communicatively coupled with one or more servers through one or more computing devices,
the ECG device comprising:
a housing; and
a plurality of leads extending from the housing and each including a coupler configured to releasably attach to an electrode; and
one or more user interfaces accessible through one of a browser and a software application installed on the one or more computing devices, the one or more computing devices at least intermittently communicatively coupled with the one or more servers and with the ECG device, the one or more user interfaces comprising at least one of:
an indicator configured to indicate a status of a communicative connection between the ECG device and the one or more servers; and
an indicator configured to indicate a status of a communicative connection between the ECG device and the one or more computing devices.

11. The system of claim 10, wherein the one or more user interfaces further comprise an indicator configured to indicate a status of a heart-related study of a patient.

12. The system of claim 10, wherein the one or more user interfaces further comprise an indicator configured to indicate a connection status of an electrode.

13. The system of claim 10, wherein the ECG device further comprises a marking switch coupled with the housing.

14. The system of claim 13, wherein the one or more user interfaces further comprise a symptoms user interface displayed in response to user engagement of the marking switch.

15. The system of claim 14, wherein the symptoms user interface displays a list of symptoms and is configured to receive a selection of one or more of the listed symptoms.

16. The system of claim 10, wherein the ECG device is configured to upload recorded data, related to the cardiac-related activity, to the one or more servers through the communicative connection between the ECG device and the one or more computing devices.

17. The system of claim 10, wherein the ECG device comprises no more than three leads and is configured to monitors the cardiac-related activity via no fewer than three channels.

18. A method of use for an electrocardiogram (ECG) device, comprising:
providing an ECG device configured to monitor cardiac-related activity and to record cardiac-related data, the ECG device comprising:
a housing;
a plurality of leads coupled with the housing and including snap couplers configured to attach to electrodes; and
a marking switch coupled with the housing and configured to initiate recording of a heart-related event upon being engaged by a user;
providing one or more user interfaces through one of a browser and a software application installed on one or more computing devices, the one or more user interfaces comprising one or more selectors configured to allow a clinician to:
communicatively couple the one or more computing devices with one or more servers and with the ECG device; and
initiate recording of the cardiac-related data by the ECG device.

19. The method of claim 18, further comprising, in response to a predetermined time period elapsing after initiating recording of the cardiac-related data, uploading the recorded cardiac-related data to the one or more servers through the one or more computing devices.

20. The method of claim 18 further comprising using an ECG classifier, at least partially comprised in one of the one or more servers and the ECG device, to analyze the cardiac-related data and to determine and indicate one of the following: placement in time of an R peak of a heartbeat; a type for the heartbeat; existence of an arrythmia; existence of a heart block; a diagnosis; heart rate variability (HRV) statistics; ST segment statistics; and QTC segment statistics.
